(19) United States
(12) Patent Application Publication
(10) Pub. No.: US 2021/0330286 A1
(43) Pub. Date: Oct. 28, 2021

(54) CATHETER SYSTEM

(71) Applicant: KYOCERA Corporation, Kyoto-shi, Kyoto (JP)

(72) Inventor: Seungmok LEE, Sakai-shi, Osaka (JP)

(21) Appl. No.: 17/045,451

(22) PCT Filed: Apr. 5, 2019
(86) PCT No.: PCT/JP2019/015201

§ 371 Date: Oct. 5, 2020

(30) Foreign Application Priority Data
Apr. 6, 2018 (JP) 2018-073804

(51) Int. Cl. A61B 8/12; A61B 8/00; A61B 8/08

(52) U.S. Cl. CPC A61B 8/12; A61B 8/466; A61B 8/4263; A61B 8/467; A61B 8/483

(57) ABSTRACT

One aspect of a catheter system includes a reference portion and a moving portion. The moving portion is movable along the reference portion in a longitudinal direction of the reference portion. The moving portion includes a wave transceiver unit that transmits/receives one or more types of waves. The wave transceiver unit transmits a first type of waves among the one or more types of waves in a direction intersecting with the longitudinal direction in a space inside a target object and receives the first type of waves reflected from the target object. The reference portion includes a reference region. In the reference region, one or more types of elements are located in the longitudinal direction based on a predetermined rule so that the state of reflection of a second type of waves included in the one or more types of waves varies in the longitudinal direction.

### CROSS-REFERENCE TO RELATED APPLICATION

The present application is a National Phase entry based on PCT Application No. PCT/JP2019/015201 filed on Apr. 5, 2019, entitled “CATHETER SYSTEM”, which claims the benefit of Japanese Patent Application No. 2018-073804, filed on Apr. 6, 2018, entitled “CATHETER SYSTEM”. The contents of which are incorporated by reference herein in their entirety.

### FIELD

The present disclosure relates to a system using a catheter (also referred to as a catheter system).

### BACKGROUND

For example, Percutaneous Coronary Intervention (PCI) using a catheter has been known in which a stenotic lesioned part of a coronary artery is expanded with a stent or the like.

### SUMMARY

A catheter system is disclosed. One aspect of the catheter system includes a reference portion and a moving portion. The moving portion is movable along the reference portion in a longitudinal direction of the reference portion. The moving portion includes a wave transceiver unit that transmits/receives one or more types of waves. The wave transceiver unit transmits a first type of waves among the one or more types of waves in a space inside a target object and receives the first type of waves reflected from the target object. The reference portion includes a reference region. In the reference region, one or more types of elements are located in the longitudinal direction based on a predetermined rule so that the state of reflection of a second type of waves included in the one or more types of waves varies in the longitudinal direction.

### BRIEF DESCRIPTION OF THE DRAWINGS

FIG. 1 is a diagram illustrating an example of a schematic configuration of a catheter system according to a first embodiment.

FIG. 2A is a side view illustrating an example of an outer appearance of a guide wire. FIG. 2B is a cross-sectional view illustrating an example of a cross-sectional schematic diagram of the guide wire taken along line IIb-IIb in FIG. 2A.

FIG. 3A is a side view illustrating an example of an outer appearance of a catheter portion in a region IIIa illustrated in FIG. 1. FIG. 3B is a diagram illustrating an example of a cross-sectional schematic diagram of the catheter portion taken along line IIIb-IIIb in FIG. 3A.

FIG. 4 is a diagram illustrating an example of a cross-sectional schematic diagram of the catheter portion taken along line IV-IV in FIG. 3A.

FIG. 5A is a diagram illustrating a part of the catheter portion inserted into a blood vessel. FIG. 5B is a diagram illustrating an example of movement of the catheter portion when the catheter portion inserted in the blood vessel is pulled.

FIG. 6A is a diagram illustrating an example of a cross-sectional schematic diagram of the catheter portion according to the first embodiment illustrating a second wave transceiver and a portion in the vicinity of the second wave transceiver, taken along line IIIb-IIIb in FIG. 3A. FIG. 6B is a diagram illustrating an example of a change in the intensity of light received by a light receiver over time.

FIG. 7A is a block diagram illustrating an example of a functional configuration of the catheter system. FIG. 7B is a block diagram illustrating a functional configuration implemented by a calculation unit.

FIG. 8 is a diagram illustrating a concept of a three-dimensional reconstruction process using a plurality of tomographic images.

FIG. 9 is a flowchart illustrating an example of operation of the catheter system according to the first embodiment.

FIG. 10A is a diagram illustrating an example of a cross-sectional schematic diagram of a catheter portion according to a second embodiment illustrating a second wave transceiver and a portion in the vicinity of the second wave transceiver, corresponding to the cross-sectional schematic diagram in FIG. 6A. FIG. 10B is a diagram illustrating another example of a cross-sectional schematic diagram of the catheter portion according to the second embodiment illustrating the second wave transceiver and a portion in the vicinity of the second wave transceiver, corresponding to the cross-sectional schematic diagram in FIG. 6A.

FIG. 11 is a diagram illustrating an example of a schematic configuration of a catheter system according to a third embodiment.

FIG. 12A is a side view illustrating an example of an outer appearance of the catheter portion in a region XIIa illustrated in FIG. 11. FIG. 12B is a diagram illustrating an example of a cross-sectional schematic diagram of the catheter portion taken along line XIIb-XIIb in FIG. 12A.

FIG. 13A is a diagram illustrating an example of a cross-sectional schematic diagram of a catheter portion according to a third embodiment illustrating a second wave transceiver and a portion in the vicinity of the second wave transceiver, taken along line XIIb-XIIb in FIG. 12A. FIG. 13B is a diagram illustrating another example of a cross-sectional schematic diagram of the catheter portion according to the third embodiment illustrating the second wave transceiver and a portion in the vicinity of the second wave transceiver, corresponding to the cross-sectional schematic diagram taken along line XIIb-XIIb in FIG. 12A.

FIG. 14 is a diagram illustrating still another example of a cross-sectional schematic diagram of the catheter portion according to the third embodiment illustrating the second wave transceiver and a portion in the vicinity of the second wave transceiver, corresponding to the cross-sectional schematic diagram taken along line XIIb-XIIb in FIG. 12A.

FIG. 15A is a diagram illustrating an example of a cross-sectional schematic diagram of a catheter portion according to a fourth embodiment illustrating a first wave transceiver and a portion in the vicinity of the first wave transceiver, corresponding to the cross-sectional schematic diagram taken along line XIIb-XIIb in FIG. 12A. FIG. 15B is a diagram illustrating another example of a cross-sectional schematic diagram of the catheter portion according to the fourth embodiment illustrating the first wave transceiver and a portion in the vicinity of the first wave transceiver, corresponding to the cross-sectional schematic diagram taken along line XIIb-XIIb in FIG. 12A.

FIG. 16A is a diagram illustrating still another example of a cross-sectional schematic diagram of the catheter portion according to the fourth embodiment illustrating the first wave transceiver and a portion in the vicinity of the first wave transceiver, corresponding to the cross-sectional schematic diagram taken along line XIIb-XIIb in FIG. 12A. FIG. 16B is a diagram illustrating yet another example of a cross-sectional schematic diagram of the catheter portion according to the fourth embodiment illustrating the first wave transceiver and a portion in the vicinity of the first wave transceiver, corresponding to the cross-sectional schematic diagram taken along line XIIb-XIIb in FIG. 12A.

FIG. 17 is a block diagram illustrating a functional configuration implemented by a calculation unit according to the fourth embodiment.

FIG. 18 is a flowchart illustrating an example of operation of a catheter system according to the fourth embodiment.

FIG. 19 is a block diagram illustrating an example of a configuration of an optical interference unit for implementing optical coherence tomography according to a fifth embodiment.

### DETAILED DESCRIPTION

An operation of Percutaneous Coronary Intervention (PCI) using a catheter involves positioning of a stent performed while capturing an X-ray image of a blood flow with a contrast agent injected into a blood vessel, for example. However, a detailed structure of the blood vessel including plaque cannot be confirmed.

An example of a catheter system to address this includes what is also known as an intravascular ultrasound (IVUS) system capable of implementing IVUS using a catheter with which a tomographic image of a blood vessel including plaque in the blood vessel can be captured. With this IVUS system, for example, data on a three-dimensional structure of a blood vessel can be obtained by reconstruction using a plurality of two-dimensional tomographic images captured in time sequence through constant speed movement of a wave transceiver unit that transmits and receives ultrasonic waves within the blood vessel. This enables a user to recognize the detailed structure of the lesioned part including the plaque structure, before and after the implementation of the PCI, for example. The detailed structure of the lesioned part includes, for example, the thickness, the inner diameter, and the outer diameter of the plaque, as well as the length of a section including the plaque.

As with the IVUS system, tomographic images of a blood vessel including plaque in the blood vessel can also be obtained using a catheter system known as an Optical Coherence Tomography (OCT) system that implements OCT using near-infrared rays, for example. Also with this OCT system, data on a three-dimensional structure of a blood vessel can be acquired by reconstruction using a plurality of tomographic images captured in time sequence through constant speed movement of a wave transceiver unit that emits and receives near-infrared rays.

To make the catheter provided with the wave transceiver unit move at a constant speed inside the blood vessel, for example, the catheter may be connected to a driving mechanism capable of pulling the catheter at a constant speed, outside the blood vessel.

Unfortunately, this may require cumbersome preparation work before the operation of the catheter system can be started. Specific examples of such work include: taking out the driving mechanism from a sterile package; attaching a guide wire to the driving mechanism; connecting the catheter to the driving mechanism; performing initial setting for the driving mechanism; and the like. As a result, it is difficult to shorten the time required for surgery, for example. For example, when the data on the three-dimensional structure of a blood vessel is to be acquired for a plurality of times during a single surgery, the initial setting work and the like for the driving mechanism might be required to be performed each time the operation is executed. As a result, it becomes even more difficult to shorten the time required for surgery, for example.

Such a problem is common to, for example, catheter systems in general capable of acquiring data on a three-dimensional structure of a tubular body of a living body including blood vessels other than coronary arteries.

In view of the above, the inventors of the present application have made a technique for improving the operability of the catheter system.

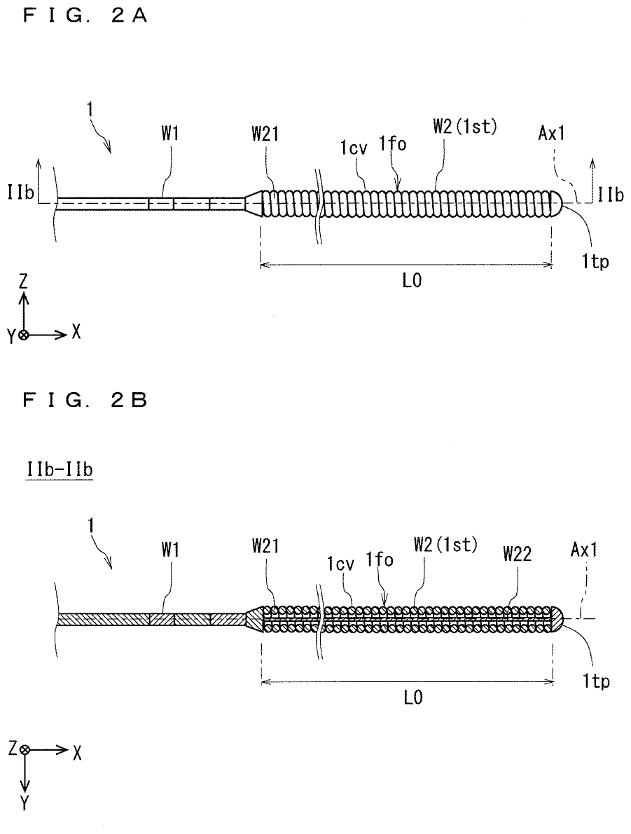

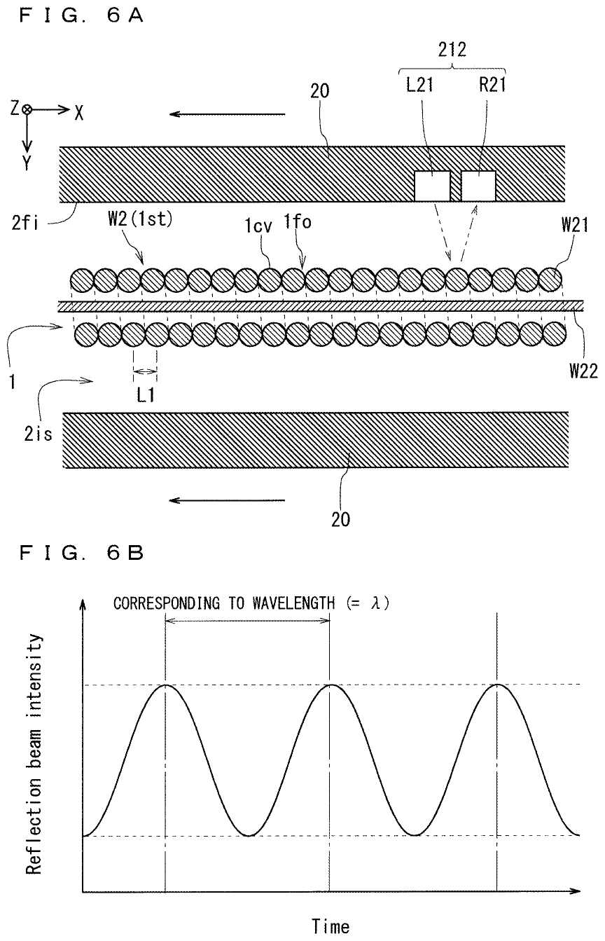

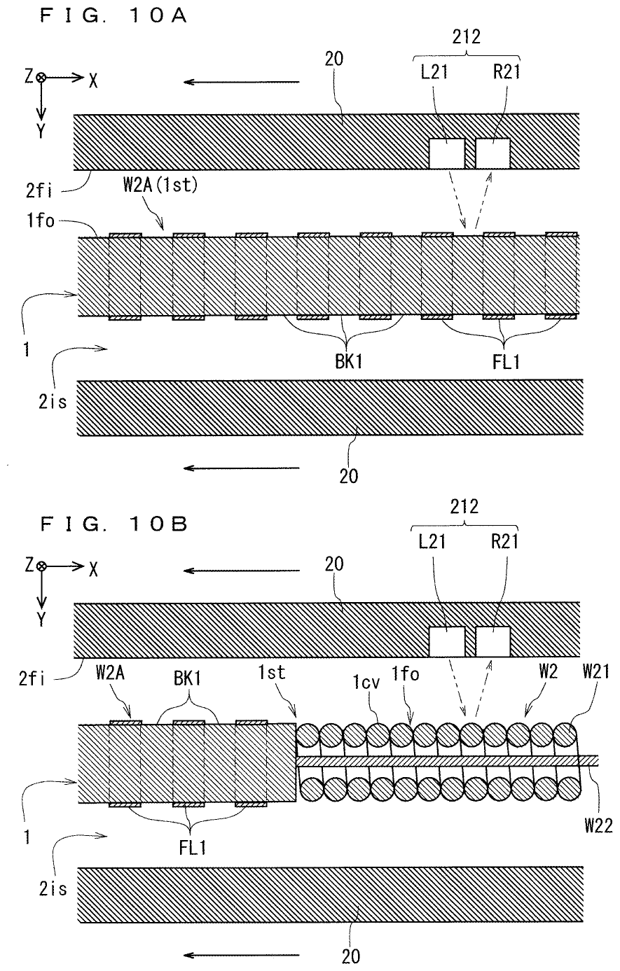

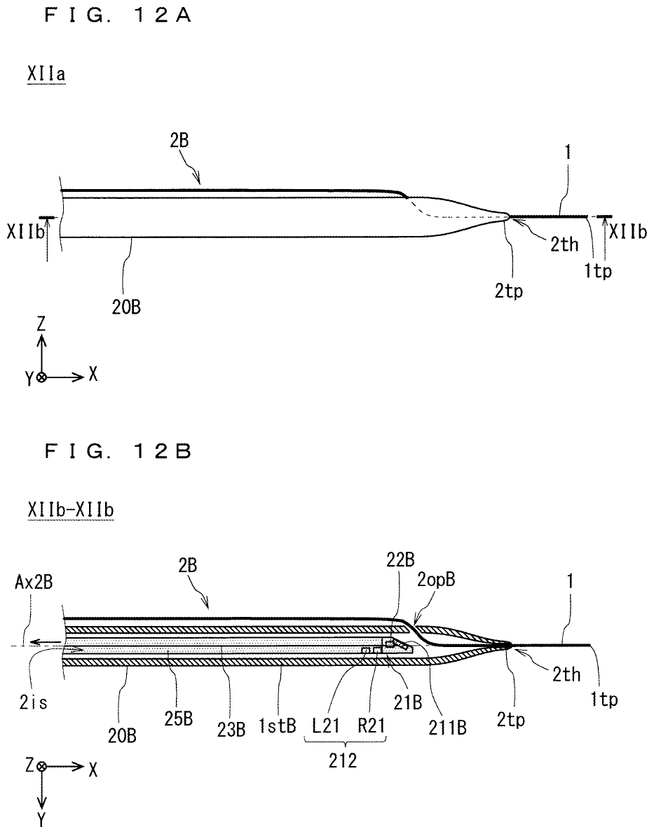

First to fifth embodiments regarding this will be described below with reference to the drawings. The same reference signs are allocated to components having similar structures and functions in the drawings, and the description thereof will not be repeated below. The drawings are illustrated schematically. FIG. 2A to FIG. 4, FIG. 6A, FIG. 6B, FIG. 10A, FIG. 10B, and FIG. 12A to FIG. 16B illustrate a right-hand XYZ coordinate system. In this XYZ coordinate system, the longitudinal direction toward a distal end 2tp of a catheter portion 2 is defined as a +X direction that is a first direction, a second direction along a radial direction of the catheter portion 2 is defined as a +Y direction, and a third direction orthogonal to both of the +X direction and the +Y direction is defined as a −Z direction.

### 1. First Embodiment

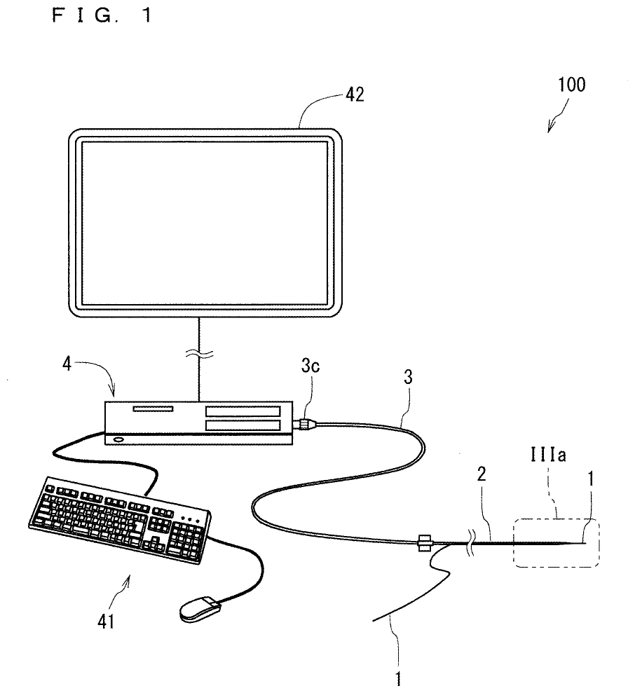

A catheter system 100 according to a first embodiment is a system using a catheter for a living body including a human body. The catheter system 100 according to the first embodiment will be described with reference to FIGS. 1 to 9.

### 1-1. Schematic Configuration of Catheter System

FIG. 1 is a diagram illustrating an example of a schematic configuration of the catheter system 100 according to the first embodiment.

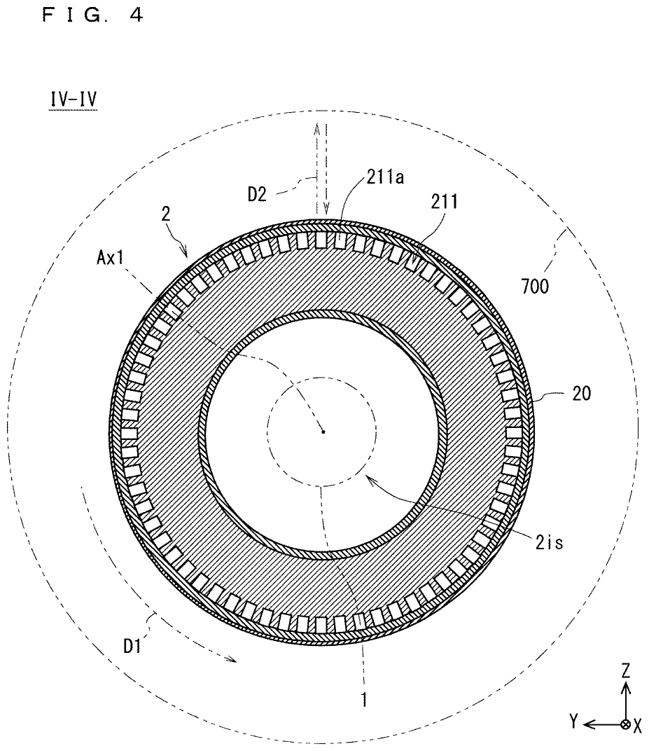

The catheter system 100 according to the first embodiment includes a guide wire 1 and the catheter portion 2. The guide wire 1 is a member for guiding the catheter portion 2 to a desired location in a meandering and curved lumen of a tubular body as a target object for processing in the living body. Here, an example of the tubular body includes a meandering and curved blood vessel 700 (see FIG. 4) and the like. Examples of the blood vessel 700 may include, a cardiac coronary artery, a cerebral blood vessel, a foot blood vessel, and the like. When the tubular body is the blood vessel 700, the lumen is the lumen of the blood vessel 700. The guide wire 1 may be made of, for example, stainless steel, a nickel titanium alloy, a platinum alloy, molybdenum, or the like. The catheter portion 2 is a thin tubular medical device with which various kinds of processing can be performed on a tubular body that is the target object. The catheter portion 2 may have any structure with a polymer formed on a substrate. Specifically, the catheter portion 2 may have any configuration with silicon, polyurethane, polyethylene, or the like formed on a glass substrate, for example.

The catheter system 100 also includes, for example, a cable portion 3 and an information processing unit 4. The information processing unit 4 can execute various kinds of information processing in the catheter system 100, for example. The cable portion 3 includes a first end portion in the longitudinal direction connected to the catheter portion 2, for example. Furthermore, the cable portion 3 includes a second end portion in the longitudinal direction provided with a connector 3c to which the information processing unit 4 is detachably attached, for example. Thus, for example, the information processing unit 4 can transmit/receive a signal to/from the catheter portion 2 through the cable portion 3. Furthermore, for example, the information processing unit 4 can supply power to the catheter portion 2 through the cable portion 3.

### 1-2. Guide Wire

The guide wire 1 is, for example, a portion (also referred to as a reference portion) that serves as a reference for the movement of the catheter portion 2. The guide wire 1 has an elongated shape.

FIG. 2A is a side view illustrating an example of an outer appearance of the guide wire 1. FIG. 2B is a cross-sectional view illustrating an example of a cross-sectional schematic diagram of the guide wire 1 taken along line IIb-IIb in FIG. 2A. In the example illustrated in FIGS. 2A and 2B, the longitudinal direction of the guide wire 1 is defined as a direction along the X axis.

The catheter system 100 according to the first embodiment employs, for example, a linear portion including a reference region 1st (also referred to as a linear guide portion) as the guide wire 1. The guide wire 1 can guide the movement of the catheter portion 2 in the longitudinal direction of the guide wire 1, for example. Here, the guide wire 1 includes a first wire portion W1 and a second wire portion W2 serving as the reference region 1st. The first wire portion W1 and the second wire portion W2 are configured to be in linearly coupled to each other in the longitudinal direction. The first wire portion W1 is used, for example, in a state of being located from the outside to the inside of the lumen of the living body. The second wire portion W2 is located on a distal end 1tp side of the guide wire 1. The second wire portion W2 (reference region 1st) is a region that serves as a reference for a change in the relative position of the catheter portion 2 with respect to the guide wire 1. In other words, in the catheter system 100, how much the position of the catheter portion 2 has changed with respect to the guide wire 1 can be determined with reference to a certain portion of the reference region 1st.

Furthermore, for example, a portion having a coil shape (also referred to as a coil-shaped portion) having a cylindrical shape formed by spirally winding a wire material W21 around a virtual axis Ax1 along the longitudinal direction is employed as the second wire portion W2. This coil-shaped portion is also referred to as a rope coil, for example. With such a second wire portion W2, for example, excellent balance between strength and flexibility in the portion of the guide wire 1 on the distal end 1tp side can be achieved. For example, high strength and flexibility can be achieved for penetrating through plaque in the blood vessel 700 without damaging the inner wall of the blood vessel 700 when the guide wire 1 is inserted into the blood vessel 700 of the living body and the distal end 1tp of the guide wire 1 is moved along the longitudinal direction of the lumen to a position beyond the lesioned part.

A length L0 of the second wire portion W2 in the longitudinal direction of the guide wire 1 may be set to any length. In the first embodiment, the length L0 is set to, for example, about 30 centimeters (cm). The outer diameter of the second wire portion W2 may be set to any diameter as long as the catheter portion 2 can be inserted into the blood vessel 700. In the first embodiment, the outer diameter of the second wire portion W2 is set to, for example, about 360 micrometers (μm). The wire material W21 of the second wire portion W2 may have any shape and diameter as long as the second wire portion W2 enabling the catheter portion 2 to be inserted in the blood vessel 700 can be formed. In the first embodiment, for example, a round wire having a circular cross section with a diameter of about 40 μm is employed as the wire material W21. Furthermore, the second wire portion W2 may include, for example, a core wire W22 located along the virtual axis Ax1. Also with the core wire W22, for example, excellent balance between strength and flexibility in the portion of the guide wire 1 on the distal end 1tp side can be achieved.

### 1-3. Catheter Portion

The catheter portion 2 is a portion (also referred to as a moving portion) that can move along the guide wire 1 in the longitudinal direction of the guide wire 1. For example, the guide wire is inserted into a lumen of a living body, and then the catheter portion 2 can move along the guide wire to reach the lesioned part. In the first embodiment, the catheter portion 2 has, for example, an elongated shape along the longitudinal direction of the guide wire 1.

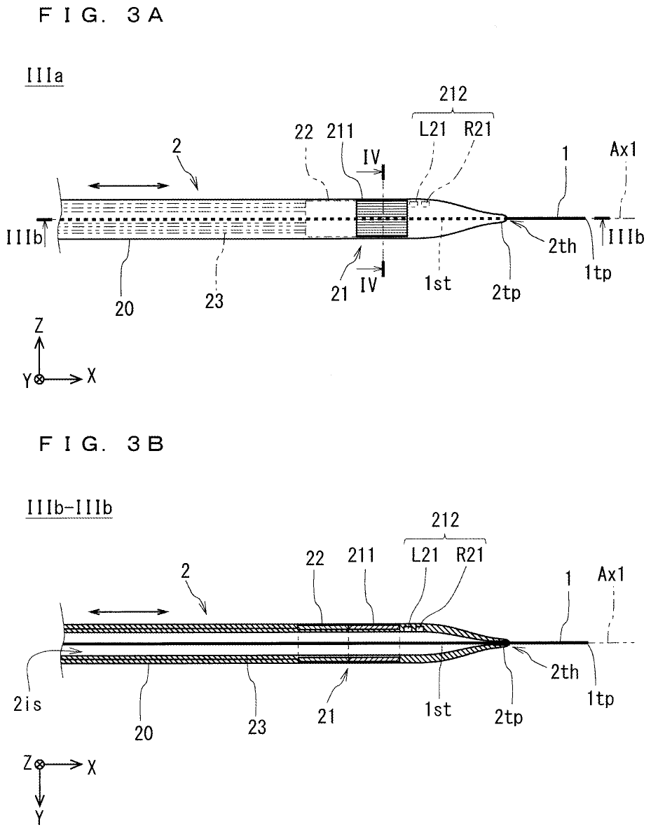

FIG. 3A is a side view illustrating an example of an outer appearance of the catheter portion 2 in a region IIIa illustrated in FIG. 1. FIG. 3B is a diagram illustrating an example of a cross-sectional schematic diagram of the catheter portion 2 taken along line IIIb-IIIb in FIG. 3A.

The catheter portion 2 includes, for example, a main body portion 20 and a wave transceiver unit 21. In the first embodiment, for example, as the catheter portion 2, a tubular portion (also referred to as a tubular moving portion) located around the guide wire 1 is employed. In other words, this tubular moving portion includes, for example, the main body portion 20 and the wave transceiver unit 21. The tubular main body portion 20 has the distal end 2tp provided with a hole portion 2th. The guide wire 1 is located from inside to outside of a lumen 2is of the main body portion 20 in a state of being inserted through the hole portion 2th. The catheter portion 2 further includes a signal processing circuit 22 and a wiring portion 23.

### 1-3-1. Wave Transceiver Unit

The wave transceiver unit 21 can transmit/receive one or more types of waves, for example. In a space on the inner side of the blood vessel 700 as the target object, the wave transceiver unit 21 can transmit a first type of waves (also referred to as first type waves) among the one or more types of waves in a direction intersecting with the longitudinal direction of the guide wire 1 serving as the reference portion, and receive the first type of waves reflected from the blood vessel 700. In the first embodiment, as illustrated in FIGS. 3A and 3B, the wave transceiver unit 21 includes, for example, a first wave transceiver 211 and a second wave transceiver 212.

### 1-3-1-1. First Wave Transceiver

The first wave transceiver 211 can, for example, transmit the first type of waves toward the blood vessel 700 and receive the first type of waves reflected from the blood vessel 700. The first wave transceiver 211 has a function of a converter that converts the received first type of waves into an electrical signal, for example. Therefore, for example, the first wave transceiver 211 can obtain a signal related to the structure of the blood vessel 700. In the first embodiment, the first wave transceiver 211 can transmit/receive ultrasonic waves, which is one type of elastic waves, as the first type of waves. Specifically, the first wave transceiver 211 can transmit/receive ultrasonic waves in a frequency band of 30 MHz to 50 MHz, for example. Thus, for example, a tomographic image related to the detailed structure of the blood vessel 700 as the target object may be easily acquired. The first wave transceiver may be manufactured by a conventionally known method.

FIG. 4 is a diagram illustrating an example of a cross-sectional schematic diagram of the catheter portion 2 taken along line IV-IV in FIG. 3A.

The first wave transceiver 211 includes a plurality of wave transceiver sections 211a. The plurality of wave transceiver sections 211a are arranged in an annular shape along a circumferential direction D1 around the guide wire 1. Each of the plurality of wave transceiver sections 211a can transmit, for example, ultrasonic waves as a first type of waves in a direction (also referred to as a separating direction) D2 away from the guide wire 1, and receive the first type of waves reflected from the blood vessel 700 as the target object. The plurality of wave transceiver sections 211a can perform transmission/reception of ultrasonic waves as the first type of waves in order along the circumferential direction D1. With the plurality of wave transceiver sections 211a arranged in an annular shape transmitting/receiving ultrasonic waves as the first type of waves over the entire circumference along the circumferential direction D1, data on a tomographic structure in one cross section of the blood vessel 700 as the target object is obtained. Thus, data on one tomographic image is obtained. In this manner, a state as if the wave transceiver sections 211a are electrically rotated is achieved without actually implementing mechanical rotation. This type of configuration may be referred to as an electronic type. Alternatively, for example, the plurality of wave transceiver sections 211a positioned over the entire circumference along the circumferential direction D1 may transmit/receive ultrasonic waves as the first type of waves in any order in time sequence, instead of transmitting/receiving waves in order along the circumferential direction D1.

When the electronic type of configuration is employed, the catheter system 100 does not require a driving mechanism for mechanically rotating the catheter portion 2. Therefore, the catheter system 100 can have a simplified configuration. Furthermore, load on the user for preparation work of the device can be reduced. As a result, the operability of the catheter system 100 may be improved.

In the catheter system 100, as the circumferential direction D1, a circumferential direction around the virtual axis Ax1 along the X axis direction as the longitudinal direction of the guide wire 1 is employed, and as the separating direction D2, a direction orthogonal to the virtual axis Ax1 is employed. The plurality of wave transceiver sections 211a are arranged in an annular shape along a virtual circle centered on the virtual axis Ax1. In the first embodiment, for example, 64 wave transceiver sections 211a are arranged in an annular shape along a circle having an outer diameter of about 1.2 millimeters (mm). Each of the plurality of wave transceiver sections 211a includes, for example, a transducer capable of transmitting/receiving ultrasonic waves, and a housing accommodating the transducer. Each of the plurality of transducers can transmit ultrasonic waves in response to a signal received from the information processing unit 4 through the cable portion 3 and the wiring portion 23, for example.

Each of the plurality of transducers can further receive, for example, the ultrasonic waves reflected from the blood vessel 700 as the target object, and output a signal corresponding to the received ultrasonic waves. The signal output from each of the plurality of wave transducers may be subjected to signal processing in the signal processing circuit 22 and may then be input to the information processing unit 4 through the wiring portion 23 and the cable portion 3. For example, in the information processing unit 4, based on a signal obtained from each of the plurality of transducers, a distance from each of the plurality of transducers to each portion of the blood vessel 700 that is the target object may be recognized in accordance with a time required for the transducer to receive the ultrasonic waves after transmitting them. Each portion may include, for example, the inner and outer walls of the blood vessel 700 and the inner wall of plaque. For example, with the plurality of wave transceiver sections 211a arranged in an annular shape transmitting/receiving ultrasonic waves, the information processing unit 4 may obtain data on a tomographic image of the blood vessel 700 that is the target object.

### 1-3-1-2. Second Wave Transceiver

The second wave transceiver 212 can, for example, transmit a second type of waves (also referred to as second type waves) among the one or more types of waves transmittable/receivable by the wave transceiver unit 21, to the reference region 1st of the guide wire 1, and can receive the second type of waves reflected from the reference region 1st. In the first embodiment, the second wave transceiver 212 can transmit/receive light, which is one type of electromagnetic waves, as the second type of waves. The wavelength of light that can be transmitted/received by the second wave transceiver 212 is not particularly limited as long as the catheter system 100 can detect the relative movement of the catheter portion 2 with respect to the guide wire 1. The second wave transceiver 212 may be manufactured by a conventionally known method.

The reference region 1st is configured, for example, to make the reflected state of the second type of waves vary in the longitudinal direction. Specifically, the reference region 1st is configured to have one or more types of elements, such as the shape, material, and color of the guide wire 1, located in the longitudinal direction according to a predetermined rule. The predetermined rule may be, for example, a pitch between one or more types of elements, a pitch between groups obtained by grouping a plurality of one or more types of elements, and the like. In the first embodiment, the predetermined rule means that one or more types of elements are located at a constant pitch in the longitudinal direction. With this configuration, how the light transmitted from the second wave transceiver 212 is reflected varies periodically, whereby the intensity of light received by the second wave transceiver 212 varies periodically. Thus, with this configuration, for example, the state of reflection of the second type of waves varies periodically as the catheter portion 2 moves with respect to the guide wire 1. Therefore, based on this periodic variation, a change in the one or more types of elements of the guide wire 1 can be detected through transmission/reception of the second type of waves by the wave transceiver unit 21. Then, based on a result of this detection, for example, the movement amount of the wave transceiver unit 21 relative to the guide wire 1, between a plurality of timings at which the wave transceiver unit 21 receives ultrasonic waves from the blood vessel 700 for obtaining a plurality of respective tomographic images of the blood vessel 700 as the target object, may be directly recognized. Thus, for example, separation distances among a plurality of portions of the blood vessel 700 captured in a plurality of respective tomographic images obtained through transmission/reception of ultrasonic waves by the wave transceiver unit 21 may be recognized. In other words, for example, to which of the portions of the blood vessel 700 the data on each of the tomographic structures at a plurality of portions is related may be more accurately recognized.

Thus, for example, data on the three-dimensional structure of the blood vessel 700 as the target object can be obtained without moving the catheter portion 2 as the moving portion at a constant speed. In other words, for example, a driving mechanism for moving the catheter portion 2 as the moving portion at a constant speed is not required. Thus, for example, preparation work and the like for a driving mechanism for moving the catheter portion 2 as the moving portion at a constant speed may be reduced. As a result, for example, the operability of the catheter system 100 for obtaining data on the three-dimensional structure of the blood vessel 700 as the target object can be improved.

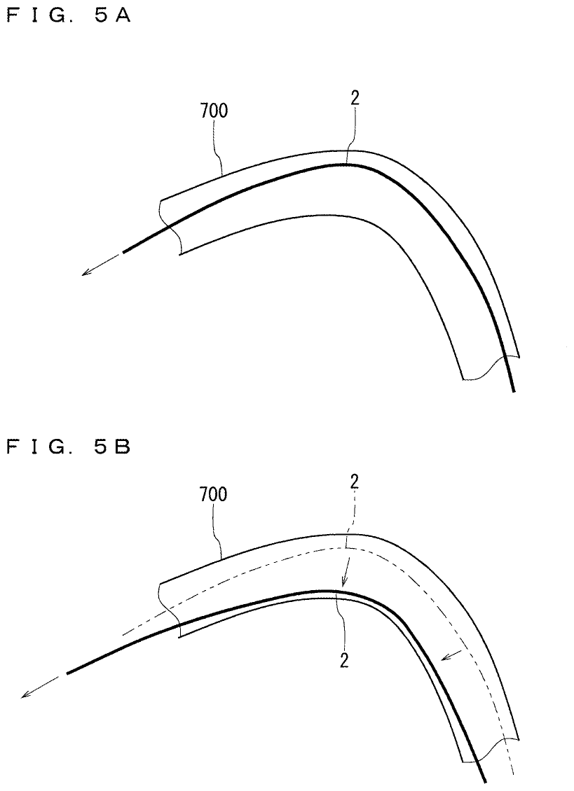

FIG. 5A is a diagram illustrating a part of the catheter portion 2 inserted into a blood vessel 700. FIG. 5B is a diagram illustrating an example of the movement of the catheter portion 2 when the catheter portion 2 inserted in the blood vessel 700 is pulled.

For example, it is assume that the catheter portion 2 is connected to a driving mechanism for moving the catheter portion 2 at a constant speed, outside the blood vessel 700. In this case, for example, as illustrated in FIG. 5A, it is first assumed that the catheter portion 2 is located in the blood vessel 700. Here, the blood vessel 700 meanders and is curved, and has an inner diameter that is larger than the outer diameter of the catheter portion 2. Therefore, for example, when the catheter portion 2 is pulled by the driving mechanism, the position of the catheter portion 2 may change in the blood vessel 700, as illustrated in FIG. 5B, for example. Here, if the catheter portion 2 is inserted into the blood vessel 700 for a long distance to reach the vicinity of the lesioned part, a change in the position of the catheter portion 2 in the blood vessel 700 occurs at a large number of portions. For example, when the lesioned part is in a coronary artery in the heart, the length of the catheter portion 2 entering the body to reach the lesioned part is about 1.5 meters (m), which is extremely long. The structure of the blood vessel 700 leading to the coronary artery in the heart is extremely complicated. Thus, for example, there may be an error between the movement distance of the catheter portion 2 recognized by the driving mechanism and the movement distance of the wave transceiver unit 21 located near the distal end 2tp of the catheter portion 2 near the lesioned part of the blood vessel 700. For example, a movement of the catheter portion 2 for about 4 to 5 cm relative to the guide wire 1 with the lesioned part being in the coronary artery in the heart may result in an error of several mm or more.

By contrast, in the catheter system 100 according to the first embodiment, for example, as described above, the intensity of the reflected light varies periodically. Thus, the movement amount of the wave transceiver unit 21 relative to the guide wire 1 may be directly recognized based on the periodical variation. Specifically, this periodic variation is based on the pitch between the one or more types of elements. Thus, the movement amount of the wave transceiver unit 21 relative to the guide wire 1 can be calculated based on the periodicity of variation, if the pitch between the one or more types of elements is known. As a result, the catheter system 100 can reduce the impact of an error between the movement distance of the catheter portion 2 and the movement distance of the wave transceiver unit 21. Thus, with the catheter system 100, for example, data on the highly accurate three-dimensional structure of the blood vessel 700 as the target object can be acquired.

FIG. 6A is a diagram illustrating an example of a cross-sectional schematic diagram of the catheter portion 2 according to the first embodiment illustrating the second wave transceiver 212 and a portion in the vicinity of the second wave transceiver 212, taken along line IIIb-IIIb in FIG. 3A. FIG. 6B is a diagram illustrating an example of a change in the intensity of light received by a light receiver R21 over time.

In the first embodiment, the second wave transceiver 212 includes, for example, a light emitter L21 and the light receiver R21. The light emitter L21 can emit light toward the reference region 1st of the guide wire 1, for example. The light receiver R21 can receive the light reflected from the reference region 1st, for example. According to a possible mode, each of the light emitter L21 and the light receiver R21 may be located on an inner circumferential portion side of the tubular main body portion 20 of the catheter portion 2, for example. In the example illustrated in FIG. 6A, the light emitter L21 and the light receiver R21 are located on an inner circumferential portion 2fi side of the tubular main body portion 20, and are arranged adjacent or close to each other in the longitudinal direction of the guide wire 1. The light emitter L21 may adopt a configuration including, for example, a small light emitting diode (LED) and a small optical system concentrating light from this LED. The light receiver R21 may adopt a photoelectric conversion element such as a photodiode, for example. For example, depending on the configuration of the small light emitting diode, the light emitter L21 may adopt a configuration without any optical system.

For example, the reference region 1st may adopt a configuration in which an outer circumferential portion 1fo has a shape based on a predetermined rule in the longitudinal direction of the guide wire 1. If this configuration is adopted, for example, when the catheter portion 2 is moved relative to the guide wire 1, the movement amount of the wave transceiver unit 21 relative to the guide wire 1 may be recognized based on variation of the state of reflection of light from the reference region 1st. Thus, with a relatively simple configuration, for example, the movement amount of the wave transceiver unit 21 relative to the guide wire 1, between a plurality of timings at which the wave transceiver unit 21 receives ultrasonic waves from the blood vessel 700 for obtaining a plurality of respective tomographic images of the blood vessel 700, may be quantitatively recognized.

In the first embodiment, for example, the outer circumferential portion 1fo of the reference region 1st includes a plurality of curved portions 1cv. The plurality of curved portions 1cv are arranged in the longitudinal direction of the guide wire 1, based on a predetermined rule, in the second wire portion W2 as the coil-shaped portion. In the example illustrated in FIG. 6A, the plurality of curved portions 1cv adopt a shape, in the longitudinal direction of the guide wire 1, defined by semicircular portions adjacently arranged at a pitch of a distance L1 corresponding to the diameter of the wire material W21.

With such a configuration, for example, when the catheter portion 2 moves along the longitudinal direction of the guide wire 1, the light emitted from the light emitter L21 is reflected from the outer circumferential portion 1fo of the guide wire 1 at an angle changing in accordance with the unevenness of the outer circumferential portion 1fo of the reference region 1st. Furthermore, for example, the intensity of light reflected from the reference region 1st and received by the light receiver R21 also changes over time. For example, a spot of light emitted by the light emitter L21 onto the reference region 1st may have any diameter smaller than the distance L1, which is the pitch of the plurality of curved portions 1cv. As illustrated in FIG. 6B, for example, the intensity of the light received by the light receiver R21 changes like a sine wave of the frequency (f) over time. Here, a cycle (λ) of the change in the intensity of light corresponds to the distance L1 of the pitch of the plurality of curved portions 1cv in the outer circumferential portion 1fo of the reference region 1st. Thus, a speed (v) of movement of the catheter portion 2 along the longitudinal direction of the guide wire 1 can be calculated by the following Formula (1), for example.

v=f·λ (1)

Thus, for example, the movement amount of the wave transceiver unit 21 relative to the guide wire 1 may be easily recognized, by using variation of the state of reflection of light on the unevenness of the outer circumferential portion 1fo of the reference region 1st included in the guide wire 1 guiding the movement of the catheter portion 2. For example, when the diameter of the curved portion 1cv of the second wire portion W2 is 40 μm, a movement distance d may be calculated by the following Formula (2) where n is the number of waves illustrated in FIG. 6B.

d=40·n (2)

### 1-4. Cable Portion

The cable portion 3 is in a state of connecting between the catheter portion 2 and the information processing unit 4 to enable transmission/reception of electrical signals between the catheter portion 2 and the information processing unit 4, for example. The cable portion 3 may be, for example, any portion formed by coating the circumference of a plurality of wire materials such as metal wires with insulating resin. The cable portion 3 adopts a structure with appropriate flexibility so as not to involve resistance against the movement of the catheter portion 2 relative to the guide wire 1, for example.

### 1-5. Information Processing Unit

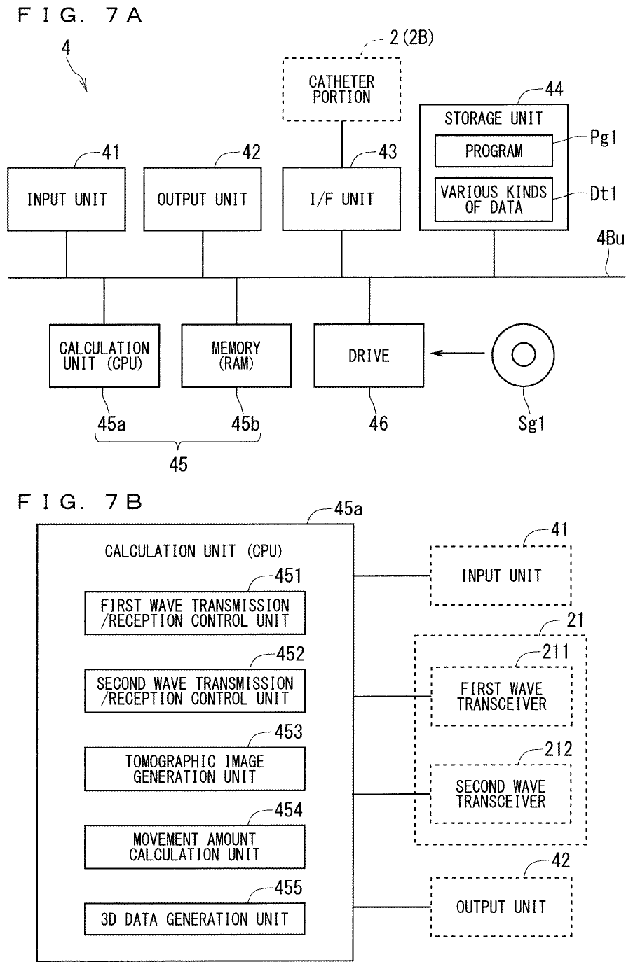

As illustrated in FIG. 7A, the information processing unit 4 includes, for example, an input unit 41, an output unit 42, an interface (I/F) unit 43, a storage unit 44, a control unit 45, and a drive 46. These components are in a state of being connected to each other by a bus line 4Bu, for example.

The input unit 41 can input, for example, a signal corresponding to an action or the like made by a user using the information processing unit 4. The input unit 41 may include, for example, an operation unit, a microphone, various sensors, and the like. The operation unit may include a mouse, a keyboard and the like that can input a signal corresponding to an operation made by the user. The microphone can input a signal corresponding to the voice of the user. The various sensors can input signals corresponding to the movement of the user. For example, the input unit 41 can input a first signal for starting the operation (also referred to as a 3D data acquisition operation) of acquiring data on the three-dimensional structure of the blood vessel 700 in the catheter system 100, in response to a first action made by the user. Furthermore, for example, the input unit 41 can input a second signal for terminating the 3D data acquisition operation in the catheter system 100 in response to a second action made by the user.

The output unit 42 can output various kinds of information, for example. The output unit 42 may include, for example, a display unit and a speaker. The display unit can, for example, visually output various kinds of information in a form recognizable by the user. The display unit may be in a form of a touch panel integrated with the input unit 41. The speaker can, for example, audibly output various kinds of information in a form recognizable by the user.

The I/F unit 43 can be connected to the catheter portion 2 to enable transmission/reception of signals through the cable portion 3.

The storage unit 44 can store various kinds of information, for example. The storage unit 44 can be configured by a storage medium such as a hard disk and a flash memory, for example. For example, the storage unit 44 may adopt any one of a configuration including one storage medium, a configuration including two or more storage media integrated, and a configuration including two or more storage media separately in two or more parts. The storage unit 44 may store, for example, a program Pg1, various kinds of data Dt1, and the like. The storage unit 44 may include a memory 45b described later.

The control unit 45 can comprehensively manage the operation of the catheter system 100 by controlling other components of the catheter system 100. The control unit 45 can also be referred to as a control device or a control circuit. The control unit 45 includes at least one processor to provide control and processing abilities to perform various functions, as described more in detail later.

According to various embodiments, at least one processor may be implemented as a single integrated circuit (IC) or as a plurality of communicatively connected integrated circuits ICs and/or discrete circuits. At least one processor may be implemented according to various known techniques.

In one embodiment, the processor includes one or more circuits or units configured to perform one or more data calculation procedures or processes, by executing instructions stored in associated memory, for example. In other embodiments, the processor may be firmware (discrete logic component, for example) configured to perform one or more data calculation procedures or processes.

According to various embodiments, the processor includes one or more processors, controllers, microprocessors, microcontrollers, application specific integrated circuits (ASICs), digital signal processors, programmable logic devices, field programmable gate arrays, any combination of these devices or configurations, or a combination of other known devices and configurations, and may execute the functions described later.

FIG. 7A is a block diagram illustrating an example of a functional configuration of the catheter system 100.

The control unit 45 includes a calculation unit 45a and a memory 45b. For example, the calculation unit 45a adopts a central processing unit (CPU). The memory 45b adopts, for example, a transitory recording medium such as a random access memory (RAM) or a non-transitory recording medium such as a read only memory (ROM) readable by the calculation unit 45a. Various functions of the control unit 45 are implemented by the calculation unit 45a executing the program Pg1 in the storage unit 44. The calculation unit 45a may include a plurality of CPUs. All or a part of the functions of the calculation unit 45a may be implemented by a hardware circuit not requiring software for implementing the function(s).

The drive 46 is, for example, a portion to which a portable storage medium Sg1 can be removably attached. In the drive 46, for example, data can be exchanged between the storage medium Sg1 and the control unit 45 while the storage medium Sg1 is attached. For example, the program Pg1 may be loaded into the storage unit 44 from the storage medium Sg1, when the storage medium Sg1 storing the program Pg1 is placed in the drive 46.

FIG. 7B is a block diagram illustrating an example of a functional configuration implemented by the calculation unit 45a including at least one processor. FIG. 7B illustrates various functions related to data processing implemented when the calculation unit 45a executes the program Pg1.

Examples of the functional configurations of the calculation unit 45a implemented include a first wave transmission/reception control unit 451, a second wave transmission/reception control unit 452, a tomographic image generation unit 453, a movement amount calculation unit 454, and a three-dimensional (3D) data generation unit 455.

The first wave transmission/reception control unit 451 can control the operation of the first wave transceiver 211, for example. For example, the first wave transmission/reception control unit 451 can cause the first wave transceiver 211 to start transmitting/receiving ultrasonic waves in response to a first signal input through the input unit 41, and cause the first wave transceiver 211 to stop transmitting/receiving the ultrasonic waves in response to a second signal input through the input unit 41.

The second wave transmission/reception control unit 452 can control the operation of the second wave transceiver 212, for example. For example, the second wave transmission/reception control unit 452 can cause the second wave transceiver 212 to start transmitting/receiving light in response to the first signal input through the input unit 41, and cause the second wave transceiver 212 to stop transmitting/receiving the light in response to the second signal input through the input unit 41.

The tomographic image generation unit 453 can generate tomographic images of the blood vessel 700 as the target object, by acquiring, for example, signals corresponding to the ultrasonic waves received by the first wave transceiver 211 via the signal processing circuit 22, the cable portion 3, and the like. For example, the tomographic image generation unit 453 can acquire a single tomographic image of the blood vessel 700 each time ultrasonic waves are transmitted/received by the plurality of wave transceiver sections 211a arranged in an annular shape, over the entire circumference along the circumferential direction D1.

The movement amount calculation unit 454 can, for example, calculate the movement amount of the wave transceiver unit 21 relative to the guide wire 1 in the longitudinal direction of the guide wire 1, in a period between first and second timings each being a timing at which the first wave transceiver 211 receives ultrasonic waves reflected from the blood vessel 700. For example, this movement amount may be calculated based on variation of the state of the light reflected from the reference region 1st and received by the second wave transceiver 212 over time, and on a predetermined rule. In the first embodiment, each of a plurality of timings including the first timing and the second timing at which the first wave transceiver 211 receives ultrasonic waves adopts a timing at which the plurality of wave transceiver sections 211a of the first wave transceiver 211 over the entire circumference along the circumferential direction D1 receive ultrasonic waves, for example. In this case, the length of a period between the plurality of timings at which the first wave transceiver 211 receives ultrasonic waves corresponds to a cycle between timings at which the plurality of wave transceiver sections 211a of the first wave transceiver 211 over the entire circumference receive ultrasonic waves. As described above, for example, the speed (v) at which the catheter portion 2 moves with respect to the guide wire 1 can be calculated by Formula (1) described above. Thus, for each period between the plurality of timings at which the first wave transceiver 211 receives ultrasonic waves, the movement amount of the wave transceiver unit 21 relative to the guide wire 1 may be calculated based on the length (time) of the period and the speed (v). This movement amount corresponds to the distance between the portions of the blood vessel 700 captured in the plurality of respective tomographic images generated in the catheter system 100. By using this distance, the data on the three-dimensional structure of the blood vessel 700 as the target object can be generated.

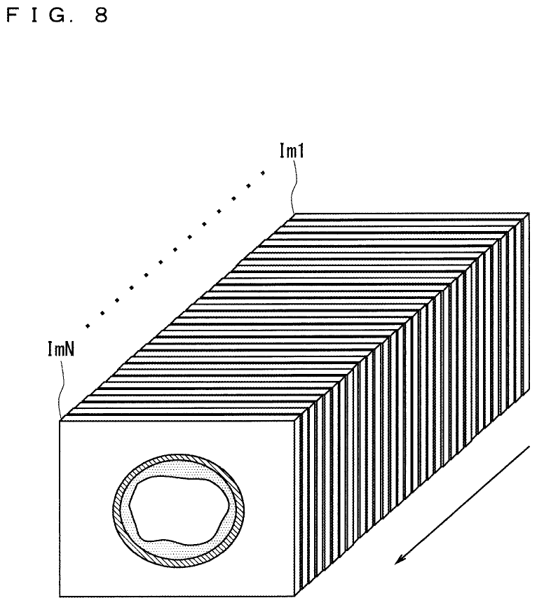

FIG. 8 is a diagram illustrating a concept of a three-dimensional reconstruction process using a plurality of tomographic images.

The 3D data generation unit 455 can generate, for example, three-dimensional data on the three-dimensional structure of the blood vessel 700. The three-dimensional data may be, for example, generated based on: a signal related to the state of the ultrasonic waves reflected from the blood vessel 700 and received by the first wave transceiver 211 at each of the plurality of timings including the first timing and the second timing at which the first wave transceiver 211 receives the ultrasonic waves reflected from the blood vessel 700; and the movement amount calculated by the movement amount calculation unit 454. The signal related to the state of the ultrasonic waves may be any signal that is output in response to the reception of ultrasonic waves by each of the plurality of transducers in the plurality of wave transceiver sections 211a. For example, the three-dimensional data may be generated with a plurality of tomographic images Im1, . . . , ImN (N being a natural number that is equal to or larger than 2) being arranged based on the movement amount between the plurality of timings. With such a configuration, for example, the data related to the three-dimensional structure of the blood vessel 700 may be easily generated.

### 1-6. Operation of Catheter System

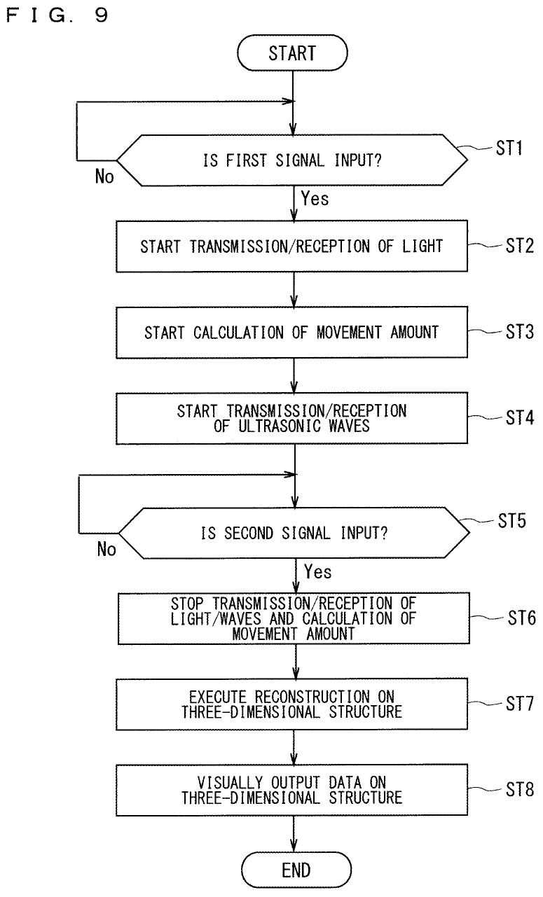

Next, the operation of the catheter system 100 will be described using an example. FIG. 9 is a flowchart illustrating an example of the operation of the catheter system 100. This operation can be implemented, for example, with the calculation unit 45a executing the program Pg1. Before this operation starts, for example, the cable portion 3 is connected to the information processing unit 4, the guide wire 1 is inserted into the blood vessel 700, and the catheter portion 2 is inserted into the blood vessel 700 along the guide wire 1. In this process, for example, the wave transceiver unit 21 of the catheter portion 2 is inserted to a position slightly beyond the lesioned part of the blood vessel 700.

FIG. 9 is a flowchart illustrating an example of operation of the catheter system 100 according to the first embodiment.

In step ST1, the calculation unit 45a determines whether or not the first signal is input through the input unit 41. The determination in step ST1 is repeated until the first signal is input. The process proceeds to step ST2 when the first signal is input.

In step ST2, the second wave transmission/reception control unit 452 causes the second wave transceiver 212 to start transmitting/receiving light. Then, the process proceeds to step ST3.

In step ST3, the movement amount calculation unit 454 starts calculation of the movement amount of the wave transceiver unit 21 relative to the guide wire 1 in the longitudinal direction of the guide wire 1. Then, the process proceeds to step ST4. In this process, the movement amount of the wave transceiver unit 21 relative to the guide wire 1 is calculated based on the signal acquired in response to the reception of light by the second wave transceiver 212.

In step ST4, the first wave transmission/reception control unit 451 causes the first wave transceiver 211 to start transmitting/receiving ultrasonic waves. Then, the process proceeds to step ST5. Thus, the signals related to the tomographic structure related to the blood vessel 700 may be sequentially acquired in response to the reception of ultrasonic waves by the first wave transceiver 211. In other words, in response to the input of the first signal through the input unit 41, the wave transceiver unit 21 starts transmitting/receiving light and ultrasonic waves as one or more types of waves.

In step ST5, the calculation unit 45a determines whether or not the second signal is input through the input unit 41. The determination in step ST5 is repeated until the second signal is input. The process proceeds to step ST6 when the second signal is input.

In step ST6, the first wave transmission/reception control unit 451 and the second wave transmission/reception control unit 452 respectively cause the first wave transceiver 211 to stop transmitting/receiving ultrasonic waves and the second wave transceiver 212 to stop transmitting/receiving light, and causes the movement amount calculation unit 454 to stop calculating the movement amount. Then, the process proceeds to step ST7. In other words, in response to the input of the second signal through the input unit 41, the wave transceiver unit 21 stops transmitting/receiving light and ultrasonic waves as one or more types of waves.

In step ST7, the 3D data generation unit 455 executes a reconstruction (also referred to as three-dimensional reconstruction) process in which three-dimensional data on the three-dimensional structure of the blood vessel 700 is generated, and the process proceeds to step ST8. In this process, the three-dimensional data may be generated based on the signals related to the states of the ultrasonic waves received by the first wave transceiver 211 and the movement amount calculated by the movement amount calculation unit 454. For example, as illustrated in FIG. 8, the three-dimensional data may be generated with the plurality of tomographic images Im1, . . . , ImN (N being a natural number that is equal to or larger than 2) being arranged based on the movement amount.

In step ST8, the calculation unit 45a visually outputs the three-dimensional data generated in step ST7 on the display unit included in the output unit 42.

In this manner, for example, the user can easily obtain the three-dimensional data on the blood vessel 700 by inputting the first signal through the input unit 41, then moving the catheter portion 2 relative to the guide wire 1 in the longitudinal direction of the guide wire 1, and then inputting the second signal through the input unit 41. Furthermore, for example, with an appropriate cross section and the like taken along the longitudinal direction of the blood vessel 700 displayed on the display unit when the three-dimensional data is visually output on the output unit 42, the user can not only obtain the structure of the blood vessel 700 but also obtain detail information such as the thickness, the inner diameter, and the outer diameter of plaque as well as the length of a section including the plaque. Furthermore, for example, the three-dimensional data on the blood vessel 700 can be obtained by the simple operation as described above, whereby the operation of obtaining the three-dimensional data can be easily performed repeatedly during a surgery.

### 1-7. Summary of First Embodiment

As described above, in the catheter system 100 according to the first embodiment, for example, when the catheter portion 2 is moved along the guide wire 1 in the longitudinal direction of the guide wire 1, the variation of the shape of the guide wire 1 in the longitudinal direction of the guide wire 1 based on the predetermined rule can be detected through emission and reception of light by the second wave transceiver 212. Thus, for example, the movement amount of the wave transceiver unit 21 relative to the guide wire 1, between a plurality of timings at which the wave transceiver unit 21 receives ultrasonic waves from the blood vessel 700 for obtaining tomographic images of the blood vessel 700, may be calculated. Thus, for example, a driving mechanism for moving the catheter portion 2 at a constant speed is not required, whereby preparation work for the driving mechanism and the like may be reduced. As a result, for example, the operability of the catheter system 100 for obtaining data on the three-dimensional structure of the blood vessel 700 may be improved.

Furthermore, for example, the movement amount of the wave transceiver unit 21 relative to the guide wire 1 may be directly recognized. Thus, to which of the portions of the blood vessel 700 the data on each of the tomographic structures at a plurality of portions is related may be more accurately recognized, for example.

### 2. Other Embodiments

The present disclosure is not limited to the first embodiment described above, and various modifications, improvements, and the like can be made without departing from the gist of the present disclosure.

### 2-1. Second Embodiment

For example, the second wire portion W2 serving as the reference region 1st in the first embodiment may be replaced with a second wire portion W2A having a different configuration.

FIG. 10A is a diagram illustrating an example of a cross-sectional schematic diagram of the catheter portion 2 according to a second embodiment illustrating the second wave transceiver 212 and a portion in the vicinity of the second wave transceiver 212, corresponding to the cross-sectional schematic diagram in FIG. 6A. FIG. 10B is a diagram illustrating another example of a cross-sectional schematic diagram of the catheter portion 2 according to the second embodiment illustrating the second wave transceiver 212 and a portion in the vicinity of the second wave transceiver 212, corresponding to the cross-sectional schematic diagram in FIG. 6A.

The outer circumferential portion 1fo of the second wire portion W2A includes a plurality of thin film portions FL1 located in the longitudinal direction of the guide wire 1 based on a predetermined rule. In the example illustrated in FIG. 10A, in the reference region 1st, the plurality of thin film portions FL1 are located in a striped pattern on the outer circumferential portion 1fo of the guide wire 1. With the main body portion of the guide wire 1 and the plurality of thin film portions FL1 made of different materials, the reflectance of light can vary between the outer circumferential portion of the main body portion of the guide wire 1 and the surfaces of the plurality of thin film portions FL1. In this case, in the outer circumferential portion 1fo of the second wire portion W2A, the states of reflection of light as the second type of waves differ between the plurality of thin film portions FL1 and portions BK1 (also referred to as blank portions) between adjacent ones of the plurality of thin film portions FL1. In other words, the reference region 1st is, for example, in a state where the reflectance of light (also referred to as light reflectance) is set to change in the longitudinal direction of the guide wire 1, based on a predetermined rule. With such a configuration adopted, for example, the movement amount of the wave transceiver unit 21 relative to the guide wire 1 may be easily recognized with a relatively simple configuration. Here, the change in the light reflectance may also be achieved by means of other elements of the outer circumferential portion 1fo of the guide wire 1 such as surface roughness, for example.

Furthermore, for example, in the reference region 1st, at least one of the shape and the light reflectance in the outer circumferential portion 1fo may be set to change in the longitudinal direction of the guide wire 1, based on a predetermined rule. If this configuration is also adopted, for example, when the catheter portion 2 is moved relative to the guide wire 1, the movement amount of the wave transceiver unit 21 relative to the guide wire 1 may be recognized based on variation of the state of reflection of light from the reference region 1st. Thus, with a relatively simple configuration, for example, the movement amount of the wave transceiver unit 21 relative to the guide wire 1, between a plurality of timings at which the first wave transceiver 211 receives ultrasonic waves from the blood vessel 700 for obtaining a plurality of respective tomographic images of the blood vessel 700, may be recognized.

For example, the reference region 1st may be adopted that is in a state in which the second wire portion W2 according to the first embodiment and the second wire portion W2A according to the second embodiment, in this order from the distal end 1tp side, are coupled to each other. With such a configuration adopted, for example, even if the second wave transceiver 212 moves along the reference region 1st to a position without the second wire portion W2, the movement amount of the wave transceiver unit 21 relative to the guide wire 1 in the longitudinal direction of the guide wire 1 may be recognized with the second wire portion W2A.

### 2-2. Third Embodiment

For example, the catheter system 100 according to each of the first embodiment and the second embodiment may be additionally provided with a driving mechanism 8B and have the catheter portion 2 converted into a catheter portion 2B that is mechanically rotated by the driving mechanism 8B, to be a catheter system 100B.

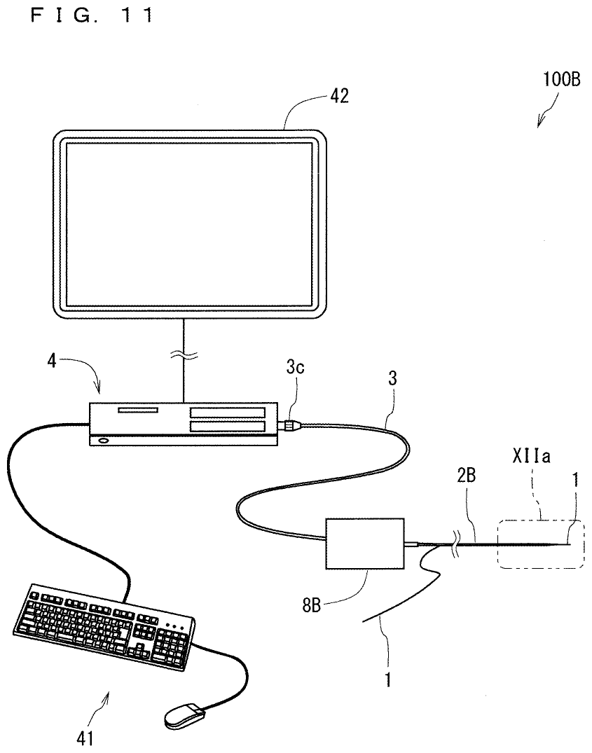

FIG. 11 is a diagram illustrating an example of a schematic configuration of the catheter system 100B according to a third embodiment. FIG. 12A is a side view illustrating an example of an outer appearance of the catheter portion 2B in a region XIIa illustrated in FIG. 11. FIG. 12B is a diagram illustrating an example of a cross-sectional schematic diagram of the catheter portion 2B taken along line XIIb-XIIb in FIG. 12A.

In the third embodiment, for example, the guide wire 1 does not serve as a reference portion, and the catheter portion 2B includes, for example, a reference portion (also referred to as a tubular reference portion) 20B having an elongated tubular shape and a sensor portion 25B serving as the moving portion. The sensor portion 25B is movable along the tubular reference portion 20B in the longitudinal direction of the tubular reference portion 20B, for example. In the example illustrated in FIGS. 12A and 12B, the sensor portion 25B has an elongated shape along the longitudinal direction of the tubular reference portion 20B. The tubular reference portion 20B is located around the sensor portion 25B, for example. The tubular reference portion 20B is a portion serving as a reference for the movement of the sensor portion 25B. The tubular reference portion 20B includes a reference region 1stB including a portion located on the distal end 2tp side in the longitudinal direction. In the examples illustrated in FIGS. 12A and 12B, the longitudinal direction of the tubular reference portion 20B is defined as a direction along the X axis. The tubular reference portion 20B has the distal end 2tp provided with the hole portion 2th. The guide wire 1 is inserted into the lumen 2is of the tubular reference portion 20B through the hole portion 2t from the outside. Furthermore, the tubular reference portion 20B includes a hole portion 2opB. The guide wire 1 is inserted through the hole portion 2opB to be led out from the inside of the lumen 2is to the outside. With such a configuration, the tubular reference portion 20B can slide along the guide wire 1. Therefore, for example, the tubular reference portion 20B can be inserted into the blood vessel along the guide wire 1 inserted into the blood vessel. Then, for example, the sensor portion 25B can reach the lesioned part along this tubular reference portion 20B.

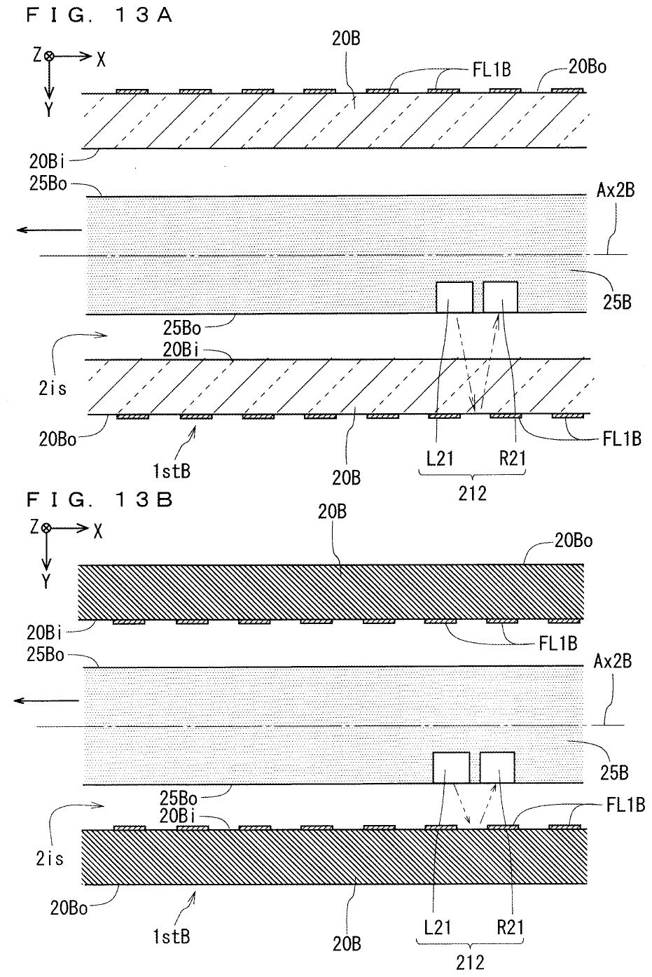

FIG. 13A is a diagram illustrating an example of a cross-sectional schematic diagram of the catheter portion 2B according to the third embodiment illustrating the second wave transceiver 212 and a portion in the vicinity of the second wave transceiver 212, taken along line XIIb-XIIb in FIG. 12A. FIG. 13B is a diagram illustrating another example of a cross-sectional schematic diagram of the catheter portion 2B according to the third embodiment illustrating the second wave transceiver 212 and a portion in the vicinity of the second wave transceiver 212, corresponding to the cross-sectional schematic diagram taken along line XIIb-XIIb in FIG. 12A.

In the reference region 1stB, for example, as in the reference region 1st according to each of the first embodiment and the second embodiment, one or more types of elements are located in the longitudinal direction of the tubular reference portion 20B based on a predetermined rule. For example, when the tubular reference portion 20B is made of transparent resin or the like, a configuration may be adopted in which an outer circumferential portion 20Bo of the tubular reference portion 20B is provided with a plurality of thin film portions FL1B arranged in the longitudinal direction of the tubular reference portion 20B based on a predetermined rule. In the example illustrated in FIG. 13A, a configuration is adopted in which the plurality of thin film portions FL1B are arranged in a striped pattern on the outer circumferential portion 20Bo of the tubular reference portion 20B. With the main body of the tubular reference portion 20B and the plurality of thin film portions FL1B made of different materials, in the outer circumferential portion 20Bo, the reflectance of light that is the second type of waves can vary between the outer circumferential portion of the main body of the tubular reference portion 20B and the surfaces of the plurality of thin film portions FL1B.

The sensor portion 25B includes, for example, a wave transceiver unit 21B. In the example illustrated in FIG. 12B, the wave transceiver unit 21B is located on the distal end side of the sensor portion 25B. The wave transceiver unit 21B includes a first wave transceiver 211B and the second wave transceiver 212. Furthermore, the sensor portion 25B includes, for example, a signal processing circuit 22B and a wiring portion 23B.

The first wave transceiver 211B is similar to the first wave transceiver 211 according to each of the first embodiment and the second embodiment, in that it can, for example, transmit ultrasonic waves as the first type of waves toward the blood vessel 700 and receive the ultrasonic waves, as the first type of waves reflected from the blood vessel 700. The first wave transceiver 211B has a function of a converter that converts the received ultrasonic waves as the first type of waves into an electrical signal, for example. The first wave transceiver 211B includes a transducer capable of transmitting/receiving ultrasonic waves, and a housing accommodating the transducer, for example.

The sensor portion 25B may be rotated by the driving mechanism 8B about a virtual axis of rotation (also referred to as a virtual rotation axis) Ax2B along the longitudinal direction of the sensor portion 25B. For example, the transducer in the first wave transceiver 211B is capable of transmitting ultrasonic waves in a direction intersecting with the virtual rotation axis Ax2B, in response to a signal input from the information processing unit 4 through the cable portion 3 and the wiring portion 23B. For example, a signal related to the tomographic structure of one portion of the blood vessel 700 as a target object can be obtained with the first wave transceiver 211B makes one rotation about the virtual rotation axis Ax2B. Such a configuration in which the first wave transceiver 211B thus mechanically rotates is referred to as a mechanical type. With the catheter system 100B adopting such a mechanical configuration, for example, the structure of the first wave transceiver 211B can be simplified. Thus, for example, the configuration of the sensor portion 25B may be simplified.

For example, the second wave transceiver 212 is located on the outer circumferential portion 25Bo side of the sensor portion 25B.

In the reference region 1stB, one or more types of elements are arranged in the longitudinal direction of the tubular reference portion 20B based on a predetermined rule, so that the state of reflection of light as the second type of waves transmitted from the second wave transceiver 212 changes in the longitudinal direction of the tubular reference portion 20B. Therefore, for example, when the sensor portion 25B is moved relative to the tubular reference portion 20B, one or more types of elements arranged in the tubular reference portion 20B based on a predetermined rule may be detected through transmission/reception of light as the second type of waves by the second wave transceiver 212. Then, based on a result of this detection, for example, the movement amount of the wave transceiver unit 21B relative to the tubular reference portion 20B, between a plurality of timings at which the wave transceiver unit 21B receives ultrasonic waves from the blood vessel 700 for obtaining a plurality of respective tomographic images of the blood vessel 700 as the target object, may be quantitatively recognized. In the third embodiment, each timing at which the first wave transceiver 211B receives ultrasonic waves is, for example, a timing at which the first wave transceiver 211B makes one rotation about the virtual rotation axis Ax2B. Thus, for example, separation distances among a plurality of portions of the blood vessel 700 captured in a plurality of respective tomographic images obtained through transmission/reception of ultrasonic waves by the wave transceiver unit 21B may be recognized.

Thus, for example, the movement amount of the wave transceiver unit 21B including the first wave transceiver 211B relative to the blood vessel 700 as the target object can be measured with high accuracy. In existing mechanical type catheter systems, for example, a motor for movement in the longitudinal direction of the sensor portion 25B and a motor for rotating the sensor portion 25B are positioned at positions outside the body, where the driving mechanism 8B is provided as illustrated in FIG. 11. Therefore, an error is likely to occur between the movement amount of the first wave transceiver 211B controlled and measured outside the body and the actual movement amount of the first wave transceiver 211B. By contrast, in the third embodiment, the measurement accuracy of the movement amount of the first wave transceiver 211B relative to the blood vessel 700 as the target object can be dramatically improved. For example, the catheter system 100B according to the third embodiment can measure the movement amount of the first wave transceiver 211B with an accuracy (unit) of 40 μm or less, even in a situation where the existing mechanical type catheter systems are only capable of measuring the movement amount of the first wave transceiver 211B with an accuracy (unit) of mm order, for obtaining data on the three-dimensional structure of the blood vessel 700 as the target object. Thus, with the catheter system 100B according to the third embodiment, for example, the data on the three-dimensional structure of the blood vessel 700 as the target object can be obtained without moving the sensor portion 25B as the moving portion at a constant speed. In other words, for example, a driving mechanism for moving the sensor portion 25B as the moving portion at a constant speed is not required. Thus, for example, preparation work and the like for a driving mechanism for moving the sensor portion 25B as the moving portion at a constant speed may be reduced. As a result, for example, the operability of the catheter system 100B for obtaining data on the three-dimensional structure of the blood vessel 700 as the target object may be improved.

For example, a configuration may be adopted in which an inner circumferential portion 20Bi of the tubular reference portion 20B is provided with the plurality of thin film portions FL1B arranged in the longitudinal direction of the tubular reference portion 20B based on a predetermined rule. In other words, at least one of the outer circumferential portion 20Bo and the inner circumferential portion 20Bi of the tubular reference portion 20B may include the plurality of thin film portions FL1B arranged in the longitudinal direction of the tubular reference portion 20B based on the predetermined rule. Also with such a configuration, for example, the movement amount of the wave transceiver unit 21B relative to the tubular reference portion 20B may be easily recognized with a relatively simple configuration. In the example illustrated in FIG. 13B, a configuration is adopted in which the plurality of thin film portions FL1B are arranged in a striped pattern on the inner circumferential portion 20Bi of the tubular reference portion 20B. For example, with the main body of the tubular reference portion 20B and the plurality of thin film portions FL1B made of different materials, in the inner circumferential portion 20Bi, the reflectance of light that is the second type of waves can vary between the inner circumferential portion of the main body of the tubular reference portion 20B and the surfaces of the plurality of thin film portions FL1B.

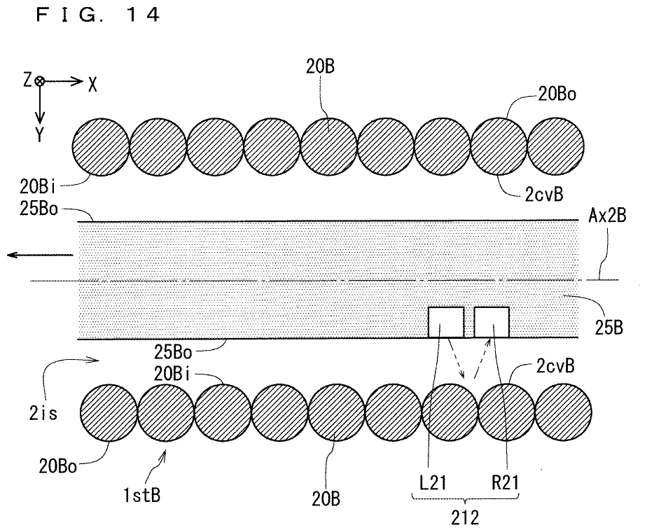

FIG. 14 is a diagram illustrating still another example of a cross-sectional schematic diagram of the catheter portion 2B according to the third embodiment illustrating the second wave transceiver 212 and a portion in the vicinity of the second wave transceiver 212, corresponding to the cross-sectional schematic diagram taken along line XIIb-XIIb in FIG. 12A.

In the reference region 1stB, at least one of the shape and the light reflectance in at least one of the inner circumferential portion 20Bi and the outer circumferential portion 20Bo may be set to change in the longitudinal direction of the tubular reference portion 20B based on a predetermined rule. With such a configuration adopted, for example, when the sensor portion 25B is moved relative to the tubular reference portion 20B, the movement amount of the wave transceiver unit 21B relative to the tubular reference portion 20B may be recognized based on variation of the state of reflection of light in the reference region 1stB. Thus, with a relatively simple configuration, for example, the movement amount of the wave transceiver unit 21B relative to the tubular reference portion 20B, between a plurality of timings at which the first wave transceiver 211B receives ultrasonic waves from the blood vessel 700 for obtaining a plurality of respective tomographic images of the blood vessel 700, may be recognized.

For example, at least one of the inner circumferential portion 20Bi and the outer circumferential portion 20Bo in the reference region 1stB of the tubular reference portion 20B may include a plurality of curved portions 2cvB arranged in the longitudinal direction of the tubular reference portion 20B based on a predetermined rule. In this case, for example, the movement amount of the wave transceiver unit 21B relative to the tubular reference portion 20B may be easily recognized, by utilizing the variation of the state of reflection of light on the unevenness of the tubular reference portion 20B. In the example illustrated in FIG. 14, the reference region 1stB adopts a portion in a form of a coil (coil-shaped portion) having a cylindrical shape formed by spirally winding a wire material around the virtual rotation axis Ax2B. For example, when the tubular reference portion 20B is made of transparent resin or the like and the outer circumferential portion 20Bo of the tubular reference portion 20B includes the plurality of curved portions 2cvB, the movement amount of the wave transceiver unit 21B relative to the tubular reference portion 20B may be easily recognized.

With the above configuration adopted, for example, the movement amount calculation unit 454 can calculate the movement amount of the wave transceiver unit 21B relative to the tubular reference portion 20B in the longitudinal direction of the tubular reference portion 20B in a period between a plurality of timings including the first timing and the second timing at which the first wave transceiver 211B of the wave transceiver unit 21B receives the ultrasonic waves reflected from the blood vessel 700. For example, this movement amount may be calculated based on variation of the state of the light reflected from the reference region 1stB and received by the second wave transceiver 212 of the wave transceiver unit 21 over time, and on a predetermined rule. In the third embodiment, the plurality of timings including the first timing and the second timing at which the first wave transceiver 211B receives ultrasonic waves adopt timings at which the first wave transceiver 211 makes one rotation about the virtual rotation axis Ax2B while transmitting/receiving the ultrasonic waves, for example. In this case, the length of each period between a plurality of timings at which the first wave transceiver 211B receives ultrasonic waves corresponds to the cycle of the rotation of the first wave transceiver 211 about the virtual rotation axis Ax2B.

### 2-3. Fourth Embodiment

In the third embodiment, for example, the wave transceiver unit 21B may have the second wave transceiver 212 omitted to be a wave transceiver unit 21C.

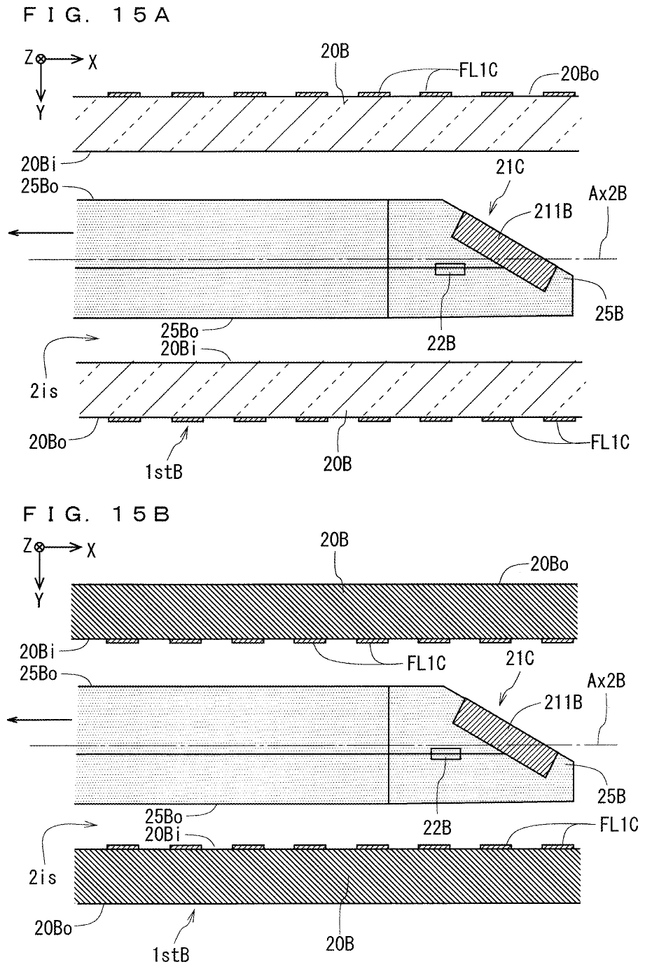

FIG. 15A is a diagram illustrating an example of a cross-sectional schematic diagram of the catheter portion 2B according to a fourth embodiment illustrating the first wave transceiver 211B and a portion in the vicinity of the first wave transceiver 211B, corresponding to the cross-sectional schematic diagram taken along line XIIb-XIIb in FIG. 12A. FIG. 15B is a diagram illustrating another example of a cross-sectional schematic diagram of the catheter portion 2B according to the fourth embodiment illustrating the first wave transceiver 211B and a portion in the vicinity of the first wave transceiver 211B, corresponding to the cross-sectional schematic diagram taken along line XIIb-XIIb in FIG. 12A.

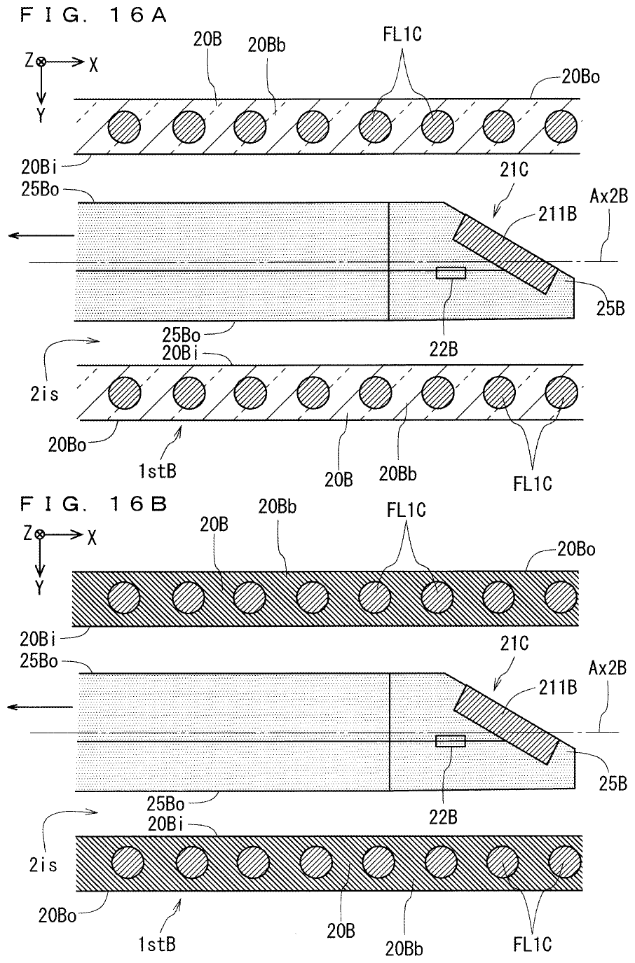

FIG. 16A is a diagram illustrating still another example of a cross-sectional schematic diagram of the catheter portion 2B according to the fourth embodiment illustrating the first wave transceiver 211B and a portion in the vicinity of the first wave transceiver 211B, corresponding to the cross-sectional schematic diagram taken along line XIIb-XIIb in FIG. 12A. FIG. 16B is a diagram illustrating yet another example of a cross-sectional schematic diagram of the catheter portion 2B according to the fourth embodiment illustrating the first wave transceiver 211B and a portion in the vicinity of the first wave transceiver 211B, corresponding to the cross-sectional schematic diagram taken along line XIIb-XIIb in FIG. 12A.

In this case, for example, a configuration may be adopted in which the first wave transceiver 211B can transmit both the first type of waves and the second type of waves toward an object, and receive both the first type of waves and the second type of waves reflected from this object. In other words, for example, a configuration may be adopted in which the first wave transceiver 211B can transmit/receive both the first type of waves and the second type of waves. Specifically, for example, in one possible configuration, ultrasonic waves that can be transmitted/received by a single transducer include the first type of waves and the second type of waves. For example, a configuration may be adopted in which the wavelength band of the first type of waves includes the wavelength band of the second type of waves. When such a configuration is adopted, for example, the first wave transceiver 211B transmits the first type of waves toward the object and receives the first type of waves reflected from the object. Thus, measurement of the movement amount of the wave transceiver unit 21C relative to the tubular reference portion 20B in the longitudinal direction of the tubular reference portion 20B and the visualization of the tomographic structure of the blood vessel 700 may be simultaneously implemented.

In this case, for example, when the distance from the first wave transceiver 211B to a later described foreign portion FL1C positioned to the tubular reference portion 20B is set in advance, a timing at which the first wave transceiver 211B receives the first type of waves reflected from the foreign portion FL1C after the first wave transceiver 211B transmits the first type of waves is within a predetermined error range. Thus, for example, a signal for visualizing the tomographic structure of the blood vessel 700 and a signal for recognizing variation of a presence/absence status of the foreign portion FL1C, obtained through transmission/reception of the first type of waves by the first wave transceiver 211B, can be distinguished from each other.

Thus, for example, through the transmission/reception of only the first type of waves by a single element of the first wave transceiver 211B, the measurement of the movement amount of the wave transceiver unit 21C relative to the tubular reference portion 20B in the longitudinal direction of the tubular reference portion 20B and visualization of the tomographic structure of the blood vessel 700 may both be implemented.

In a possible configuration in this case, for example, recognition of the movement amount of the wave transceiver unit 21C relative to the tubular reference portion 20B in the longitudinal direction of the tubular reference portion 20B and acquisition of a signal related to the tomographic structure of the blood vessel 700 as the target object may be implemented through transmission/reception of ultrasonic waves as the first type of waves. In this case, for example, the reference region 1stB may have a configuration including one or more types of elements arranged in the longitudinal direction based on a predetermined rule so that the state of reflection of the ultrasonic waves transmitted from the first wave transceiver 211B changes in the longitudinal direction. Here, for example, the shape and material may be adopted as one or more types of elements. With such a configuration adopted, for example, a special configuration may not be required for recognizing the movement amount of the wave transceiver unit 21C relative to the tubular reference portion 20B. Thus, for example, the configuration of the sensor portion 25B as the moving portion may be simplified.

In a possible configuration in this case, for example, the reference region 1stB includes a plurality of foreign portions FL1C in one or more portions on the inner circumferential portion 20Bi, an internal portion 20Bb, and the outer circumferential portion 20Bo of the tubular reference portion 20B. As the material of the foreign portion FL1C, for example, a material capable of reflecting ultrasonic waves as the first type of waves can be adopted. Furthermore, the plurality of foreign portions FL1C are, for example, located in the longitudinal direction of the tubular reference portion 20B based on a predetermined rule, and are formed of a material different from that for portions of the tubular reference portion 20B around the foreign portions FL1C. Thus, for example, the configurations of the first wave transceiver 211B and the wave transceiver unit 21C are simplified. As a result, the configuration of the sensor portion 25B as the moving portion may be simplified.

In the example illustrated in FIG. 15A, the plurality of foreign portions FL1C are located on the outer circumferential portion 20Bo of the tubular reference portion 20B. In the example illustrated in FIG. 15B, the plurality of foreign portions FL1C are located on the inner circumferential portion 20Bi of the tubular reference portion 20B. In the examples illustrated in FIG. 16A and FIG. 16B, the plurality of foreign portions FL1C are located on the internal portion 20Bb of the tubular reference portion 20B. At this time, the material of the internal portion 20Bb of the tubular reference portion 20B may be transparent or opaque, for example.

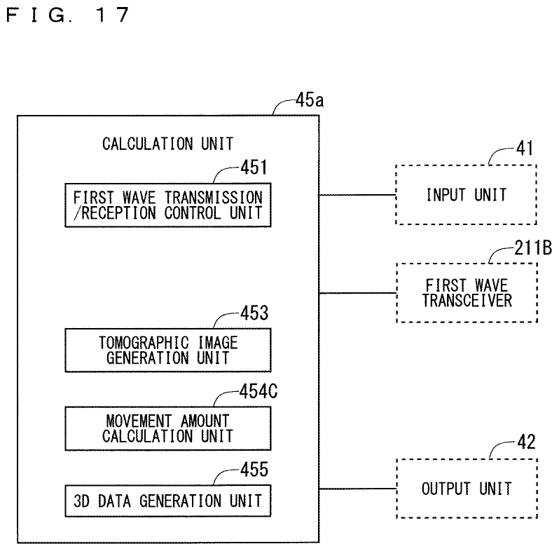

FIG. 17 is a block diagram illustrating a functional configuration implemented by the calculation unit 45a according to the fourth embodiment.

In the fourth embodiment, examples of the functional configurations of the calculation unit 45a implemented includes the first wave transmission/reception control unit 451, the tomographic image generation unit 453, a movement amount calculation unit 454C, and the 3D data generation unit 455. For example, the movement amount calculation unit 454C is similar to the movement amount calculation unit 454 according to the third embodiment in that, for example, it can calculate the movement amount of the wave transceiver unit 21B relative to the tubular reference portion 20B in the longitudinal direction of the tubular reference portion 20B in a period between the first timing and the second timing at which the first wave transceiver 211B receives the ultrasonic waves reflected from the blood vessel 700. For example, this movement amount may be calculated based on variation of the state of the ultrasonic waves reflected from the reference region 1stB and received by the first wave transceiver 211B over time, and on a predetermined rule. The plurality of timings including the first timing and the second timing at which the first wave transceiver 211B receives the ultrasonic waves are timings at which the first wave transceiver 211B makes one rotation about the virtual rotation axis Ax2B, for example, as in the third embodiment. In this case, the length of each period between a plurality of timings at which the first wave transceiver 211B receives the ultrasonic waves corresponds to the cycle of the rotation of the first wave transceiver 211B about the virtual rotation axis Ax2B.

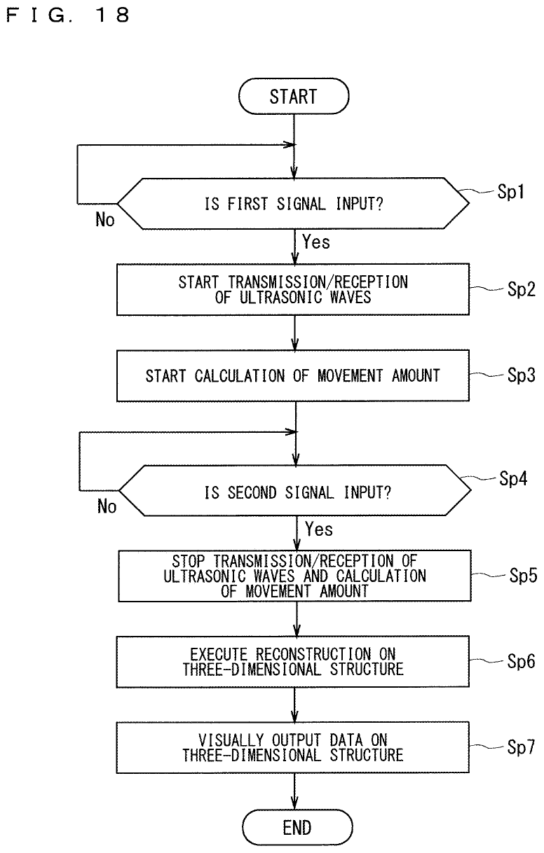

FIG. 18 is a flowchart illustrating an example of operation of the catheter system 100B according to the fourth embodiment.

This operation can be implemented, for example, with the calculation unit 45a executing the program Pg1. Before this operation starts, for example, the cable portion 3 is connected to the information processing unit 4, the catheter portion 2B is attached to the driving mechanism 8B, the guide wire 1 is inserted into the blood vessel 700, and the catheter portion 2B is inserted into the blood vessel 700 along the guide wire 1. In this process, for example, the wave transceiver unit 21C of the catheter portion 2B is inserted to a position slightly beyond the lesioned part of the blood vessel 700.

In step Sp1, the calculation unit 45a determines whether or not the first signal is input through the input unit 41. The determination in step Sp1 is repeated until the first signal is input. The process proceeds to step Sp2 when the first signal is input.

In step Sp2, the first wave transmission/reception control unit 451 causes the first wave transceiver 211B to start transmitting/receiving ultrasonic waves. Then, the process proceeds to step Sp3. In response to the input of the first signal through the input unit 41, the wave transceiver unit 21C starts transmitting/receiving ultrasonic waves as one or more types of waves.

In step Sp3, the movement amount calculation unit 454C starts calculation of the movement amount of the wave transceiver unit 21B relative to the tubular reference portion 20B in the longitudinal direction of the tubular reference portion 20B. Then, the process proceeds to step Sp4. The movement amount of the wave transceiver unit 21B relative to the tubular reference portion 20B is calculated based on a signal acquired in response to the state of the ultrasonic waves reflected from the reference region 1stB and received by the first wave transceiver 211B.

In step Sp4, the calculation unit 45a determines whether or not the second signal is input through the input unit 41. The determination in step Sp4 is repeated until the second signal is input. The process proceeds to step Sp5 when the second signal is input.

In step Sp5, the first wave transmission/reception control unit 451 causes the first wave transceiver 211B to stop transmitting/receiving ultrasonic waves and causes the movement amount calculation unit 454C to stop calculating the movement amount. Then, the process proceeds to step Sp6. In response to the input of the second signal through the input unit 41, the wave transceiver unit 21C stops transmitting/receiving ultrasonic waves as one or more types of waves.

In step Sp6, the 3D data generation unit 455 executes the three-dimensional reconstruction process in which three-dimensional data on the three-dimensional structure of the blood vessel 700 is generated, and the process proceeds to step Sp7. In this process, the three-dimensional data may be generated based on the signal related to the state of the ultrasonic waves received by the first wave transceiver 211B and the movement amount calculated by the movement amount calculation unit 454C.

In step Sp7, the calculation unit 45a visually outputs the three-dimensional data generated in step Sp6 on the display unit included in the output unit 42.

Also with this configuration, for example, the user can easily obtain the three-dimensional data on the blood vessel 700 by inputting the first signal through the input unit 41, then moving the sensor portion 25B relative to the tubular reference portion 20B in the longitudinal direction of the tubular reference portion 20B, and then inputting the second signal through the input unit 41. Furthermore, for example, when the three-dimensional data is visually output to the output unit 42, an appropriate cross section taken along the longitudinal direction of the blood vessel 700 and the like may be displayed on the display unit. Furthermore, for example, the three-dimensional data on the blood vessel 700 can be obtained by the simple operation as described above, whereby the operation of obtaining the three-dimensional data can be easily performed repeatedly during a single surgery.

In the first embodiment to the third embodiment described above, for example, the signal related to the tomographic structure of the blood vessel 700 may be obtained by the first wave transceivers 211, 211B, and the signal related to the movement amount of the wave transceiver units 21, 21B may be acquired by the second wave transceiver 212. In other words, in the wave transceiver units 21, 21B, the signal related to the tomographic structure of the blood vessel 700 and the signal related to the movement amount of the wave transceiver units 21 and 21B may be separately acquired. With such a configuration adopted, for example, the accuracy in recognizing the movement amount of the wave transceiver units 21 and 21B and the accuracy in information on the tomographic structure of the blood vessel 700 can be improved.

### 2-4. Fifth Embodiment

In each of the first embodiment to the fourth embodiment described above, for example, as the first type of waves transmitted/received by the first wave transceivers 211, 211B, light including near-infrared rays may be adopted instead of ultrasonic waves. In other words, for example, in the catheter systems 100, 100B according to each of the first embodiment to the fourth embodiment described above, instead of implementing the intravascular ultrasound (IVUS), optical coherence tomography (OCT) may be implemented. In other words, the catheter portion 2 and the sensor portion 25B as the moving portion may transmit/receive one or more types of waves including elastic waves such as ultrasonic waves and electromagnetic waves such as visible rays or infrared rays. Also with such a configuration adopted, for example, tomographic images related to the detailed structure of the blood vessel 700 as the target object may be easily acquired.

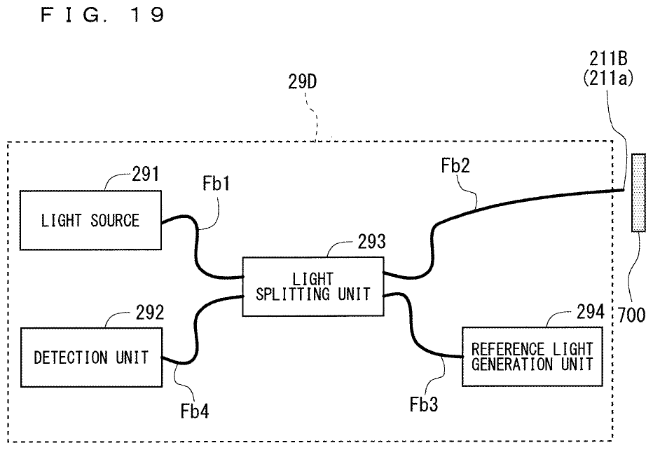

FIG. 19 is a block diagram illustrating an example of a configuration of an optical interference unit 29D for implementing the optical coherence tomography according to the fifth embodiment.

The OCT measurement method includes a time domain (TD) type and a frequency domain (FD) type. First of all, an example in which the TD-type OCT is executed will be described. In this case, for example, the catheter systems 100, 100B adopting the optical interference unit 29D may implement TD-type OCT. The optical interference unit 29D has a configuration similar to that of an interferometer and can obtain a signal related to a detailed tomographic structure of the blood vessel 700 as the target object.

The optical interference unit 29D includes, for example, a light source 291, a detection unit 292, a light splitting unit 293, and a reference light generation unit 294. The light source 291 can emit light such as near-infrared rays by using a light emitting diode (LED) or the like, for example. The light emitted from the light source 291 is input to the light splitting unit 293 through a first optical path Fb1 such as an optical fiber, for example. The light splitting unit 293 can split the light from the light source 291 using an element such as a semitransparent mirror, for example. For example, first light of the light split by the light splitting unit 293 is sent to the first wave transceiver 211B or the wave transceiver section 211a through a second optical path Fb2 such as an optical fiber. In addition, for example, second light of the light split by the light splitting unit 293 is sent to the reference light generation unit 294 through a third optical path Fb3 such as an optical fiber. The first wave transceiver 211B or the wave transceiver section 211a can emit the first light toward the blood vessel 700 as the target object, and receive light as a result of the first light reflected from the blood vessel 700 (also referred to as third light), for example. Here, the third light received by the first wave transceiver 211B or the wave transceiver section 211a is sent to the light splitting unit 293 through the second optical path Fb2, for example. The reference light generation unit 294 can adjust an optical path length for the second light, and then return the second light for which the optical path length has been adjusted (also referred to as fourth light), to the third optical path Fb3, for example. The reference light generation unit 294 adopts a configuration of adjusting the optical path length for the second light, with the second light reflected from a reflection unit while changing the distance between the third optical path Fb3 and the reflection unit as appropriate, for example. Here, the fourth light returning from the reference light generation unit 294 to the third optical path Fb3 is sent to the light splitting unit 293 though the third optical path Fb3, for example. The light splitting unit 293 can generate fifth light by superimposing the third light and the fourth light. This fifth light is sent to the detection unit 292 through a fourth optical path Fb4 such as an optical fiber. The detection unit 292 can detect the intensity of the fifth light. By using the principle of optical interference, for example, the position of the reflection unit where constructive interference between the third light and the fourth light occurs may be observed. As a result, for example, the distance from the first wave transceiver 211B or the wave transceiver section 211a to a portion where the first light is reflected in the blood vessel 700 as the target object may be detected. In the reference light generation unit 294, for example, the fourth light may be generated by changing the phase of the second light or the like.

Next, an example in which the FD-type OCT is executed will be described. Also in this case, for example, the catheter system 100, 100B adopting the optical interference unit 29D can implement FD-type OCT. Still, the light source 291 adopts a configuration including a variable wavelength laser capable of quickly changing the wavelength of emitted light, for example. The reference light generation unit 294 adopts, for example, a configuration enabling the second light to return to the third optical path Fb3, with the second light simply reflected from the fixed reflection unit and without adjusting the optical path length for the second light. For example, the detection unit 292 detects the intensity of the fifth light, with the wavelength of the laser beam emitted from the light source 291 quickly and repeatedly changed from one end to the other end of a variable range. In this process, an interference signal is obtained by superimposing a plurality of sine waves having different amplitudes and frequencies depending on the distance from the first wave transceiver 211B or the wave transceiver section 211a to the surface and the interface of each portion of the blood vessel 700 as the target object as well as the surface of plaque. By performing Fourier transform on this interference signal by the calculation unit 45a, a relationship between the distance corresponding to the frequency of the interference signal and the reflectance corresponding to the amplitude of the interference signal is obtained. As a result, for example, the distance from the first wave transceiver 211B or the wave transceiver section 211a to a portion where the first light is reflected in the blood vessel 700 as the target object may be detected. With such FD-type OCT, for example, the time required for detecting the distance from the first wave transceiver 211B or the wave transceiver section 211a to each portion can be much shorter than that in TD-type OCT. With FD-type OCT, for example, the influence of noise can be reduced by acquiring the interference signals corresponding to the same portion for a plurality of times and then executing averaging processing. Thus, the image quality of the obtained tomographic image can be improved. Furthermore, for example, with FD-type OCT featuring a shorter time required for detecting the distance from the first wave transceiver 211B or the wave transceiver section 211a to each portion, the first wave transceivers 211, 211B can be moved faster.

The configuration related to the interferometer in the optical interference unit 29D is not limited to those described above, and a configuration related to other interferometers may be adopted.

### 3. Others

In each of the third embodiment to the fifth embodiment described above, for example, the sensor portion 25B located inside the tubular reference portion 20B may include the first wave transceiver 211 of the electronic type instead of the first wave transceiver 211B of the mechanical type.

In each of the third embodiment to the fifth embodiment described above, for example, the sensor portion 25B may be manually rotated instead of being rotated by the driving mechanism 8B. In this case, for example, with a rotation sensor that detects the rotation angle of the sensor portion 25B, the rotation angle and orientation of the wave transceiver units 21B, 21C can be detected. Thus, for example, a tomographic image can be acquired. The rotation sensor may be, for example, a non-contact-type sensor that uses magnetism, light or the like, or a contact-type sensor that uses a gear or the like.

In each of the first embodiment to the fifth embodiment described above, for example, one or more types of elements in the reference regions 1st, 1stB in the longitudinal direction may be arranged in a form other than the striped pattern, based on a predetermined rule. Such other forms may include, for example, various patterns such as dots or spirals.

In each of the first embodiment to the fifth embodiment described above, for example, in the three-dimensional reconstruction process in the 3D data generation unit 455, for each interval between a plurality of tomographic images, the pixel value may be calculated by interpolation processing or the like.

It goes without saying that all or a part of each of the first embodiment to the fifth embodiments and a plurality of modified examples can be combined as appropriate in a non-contradictory range.

1. A catheter system comprising:
a reference portion including a reference region having one or more types of elements; and
a moving portion movable along the reference portion in a longitudinal direction of the reference portion, the moving portion including a wave transceiver unit that transmits and receives one or more types of waves;
wherein the wave transceiver unit transmits a first type of waves of the one or more types of waves in a space inside a target object, and receives the first type of waves reflected from the target object, and
the one or more types of elements are located in the longitudinal direction of the reference region based on a predetermined rule so that a state of reflection of a second type of waves of the one or more types of waves varies in the longitudinal direction.

2. The catheter system according to claim 1 further comprising a calculation unit that calculates a movement amount of the wave transceiver unit, relative to the reference portion in the longitudinal direction, based on variation of the state of reflection of the second type of waves reflected from the reference region and received by the wave transceiver unit over time, and based on the predetermined rule, in a period between a first timing and a second timing during which the wave transceiver unit receives the first type of waves reflected from the target object.

3. The catheter system according to claim 2, wherein the calculation unit generates three-dimensional data on a three-dimensional structure of the target object, based on a signal related to a state of the first type of waves reflected from the target object and received by the wave transceiver unit at each of the first timing and the second timing, and based on the movement amount.

4. The catheter system according to claim 3 further comprising an input unit that inputs a first signal in response to a first action made by a user, and inputs a second signal in response to a second action made by the user, wherein
the calculation unit causes the wave transceiver unit to start transmitting and receiving the one or more types of waves in response to the first signal input through the input unit, and causes the wave transceiver unit to stop transmitting and receiving the one or more types of waves in response to the second signal input through the input unit, and generates the three-dimensional data.

5. The catheter system according to claim 1, wherein the first type of waves includes ultrasonic waves.

6. The catheter system according to claim 1, wherein the first type of waves includes near-infrared rays.

7. The catheter system according to claim 1, wherein the wave transceiver unit includes a first wave transceiver that transmits the first type of waves toward the target object and receives the first type of waves reflected from the target object, and a second wave transceiver that transmits the second type of waves toward the reference region and receives the second type of waves reflected from the reference region.

8. The catheter system according to claim 7, wherein
the reference portion includes a linear guide portion including the reference region, and
the moving portion includes a tubular moving portion including the wave transceiver unit and located around the linear guide portion.

9. The catheter system according to claim 8, wherein
the first wave transceiver includes a plurality of wave transceiver sections arranged in an annular shape along a circumferential direction around the linear guide portion,
each wave transceiver section of the plurality of wave transceiver sections transmits the first type of waves and receives the first type of waves reflected from the target object, and
the plurality of transceiver sections transmits and receives the first type of waves in time sequence.

10. The catheter system according to claim 9, wherein at least one of shape and light reflectance in an outer circumferential portion of the reference region is set to change in the longitudinal direction, based on the predetermined rule.

11. The catheter system according to claim 10, wherein
the reference region includes a coil-shaped portion having a cylindrical shape, the coil-shaped portion including a wire material that spirally winds around a virtual axis along the longitudinal direction, and
the outer circumferential portion includes a plurality of curved portions arranged in the longitudinal direction in the coil-shaped portion, based on the predetermined rule.

12. The catheter system according to claim 10, wherein the outer circumferential portion includes a plurality of thin film portions located in the longitudinal direction, based on the predetermined rule.

13. The catheter system according to claim 7, wherein the reference portion includes a tubular reference portion including the reference region and located around the moving portion.

14. The catheter system according to claim 13, wherein at least one of shape and light reflectance in at least one of an inner circumferential portion and an outer circumferential portion of the reference region is set to change in the longitudinal direction, based on the predetermined rule.

15. The catheter system according to claim 14, wherein at least one of the inner circumferential portion and the outer circumferential portion includes a plurality of curved portions arranged in the longitudinal direction, based on the predetermined rule.

16. The catheter system according to claim 14, wherein at least one of the inner circumferential portion and the outer circumferential portion includes a plurality of thin film portions located in the longitudinal direction, based on the predetermined rule.

17. The catheter system according to claim 7, wherein the second wave transceiver includes a light emitter that emits light toward the reference region, and a light receiver that receives light reflected from the reference region.

18. The catheter system according to claim 1, wherein the wave transceiver unit includes a first wave transceiver that transmits the first type of waves and the second type of waves toward an object, and receives the first type of waves and the second type of waves reflected from the object.

19. The catheter system according to claim 18, wherein
the reference portion includes a tubular reference portion located around the moving portion,
the tubular reference portion includes the reference region, and
the reference region includes a plurality of foreign portions in one or more portions on an inner circumferential portion, an internal portion, and an outer circumferential portion of the tubular reference portion, the plurality of foreign portions located in the longitudinal direction based on the predetermined rule, and a material that forms the plurality of foreign portions is different from a material that forms portions of the tubular reference portion around the plurality of foreign portions.
